(19) United States
(12) Patent Application Publication
(10) Pub. No.: US 2008/0227635 A1
(43) Pub. Date: Sep. 18, 2008

(54) Dicyano Alkanoic Acid Amides for Combating Animal Pests

(75) Inventors: Michael Hofmann, Bad Durkheim (DE); Henricus Maria Martinus Bastiaans, Usingen (DE); Jurgen Langewald, Mannheim (DE); Hassan Oloumi-Sadeghi, Raleigh, NC (US); Deborah L. Culbertson, Fuquay Varina, NC (US)

Correspondence Address: BRINKS, HOFER, GILSON & LIONE, 2801 SLATER ROAD, SUITE 120, MORRISVILLE, NC, 27560, US

(73) Assignee: BASF Aktiengesellschaft, Ludswigshafen (DE)

(21) Appl. No.: 11/997,224

(22) PCT Filed: Jul. 27, 2006
(86) PCT No.: PCT/EP06/64728

§ 371 Date: Jan. 29, 2008

Related U.S. Application Data
(60) Provisional application No. 60/704,553, filed on Aug. 1, 2005.

(51) Int. Cl. A01N 25/34; C07C 233/00; A01P 7/04; A01P 5/00; A01P 7/02; A01N 37/18

(52) U.S. Cl. 504/100; 564/192; 514/625

(57) ABSTRACT

Dicyano alkanoic acid amides of formula (I), wherein R¹ is H, alkyl, haloalkyl, alkenyl, haloalkenyl, alkynyl, or haloalkynyl; R² is H, CN, alkyl, haloalkyl, alkenyl, haloalkenyl, alkynyl, haloalkynyl, cycloalkyl, halocycloalkyl, alkoxy, or haloalkoxy; R³ and R⁴ are H, alkyl, haloalkyl, alkenyl, haloalkenyl, alkynyl, haloalkynyl, cycloalkyl, halocycloalkyl, cycloalkenyl, halocycloalkenyl, or R³ and R⁴ together can be alkylene, haloalkylene, alkenylene or haloalkenylene; X is O or S; R⁵ and R⁶ are H, alkyl, optionally substituted with 1 to 3 groups CN, NO₂, OH, NR⁷R⁸, alkoxy, haloalkoxy, alkylthio, haloalkylthio, cycloalkyl, halocycloalkyl and optionally substituted phenyl; or haloalkyl, cycloalkyl, halocycloalkyl, alkenyl, haloalkenyl, alkynyl, haloalkynyl, alkylsulfinyl, haloalkylsulfinyl, alkylsulfonyl, haloalkylsulfonyl, NR⁷R⁸, alkoxycarbonyl, haloalkoxycarbonyl, alkenyloxycarbonyl, haloalkenyloxycarbonyl, alkylcarbonyl, haloalkylcarbonyl, R⁷R⁸N—C(═O)—, phenylcarbonyl, benzylcarbonyl or phenyl wherein phenylcarbonyl, benzylcarbonyl and phenyl are optionally substituted; or R⁵ and R⁶ are a saturated, partially unsaturated or unsaturated 5- to 6-membered heterocyclic radical which may contain 1 to 4 heteroatoms selected from oxygen, nitrogen and sulfur and which radical may be bonded via a carbonyl (C(═O)) linkage, and which radical is optionally substituted; R⁷, R⁸ are H, alkyl, haloalkyl, alkylcarbonyl, haloalkylcarbonyl, benzyl, phenyl, phenylcarbonyl, alkylsulfonyl, or haloalkylsulfonyl; or the enantiomers or diastereomers or salts thereof, processes for the preparation of compounds of formula (I), use of compounds of formula (I) for combating insects, acarids, or nematodes, and a method for treating, controlling, preventing or protecting animals against infestation or infection by parasites using compounds of formula (I).

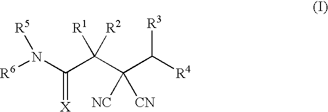

The present invention relates to dicyano alkanoic acid amides of formula I

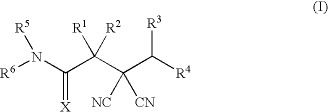

wherein
R¹ is hydrogen, C₁-C₁₀-alkyl, C₁-C₁₀-haloalkyl, C₂-C₁₀-alkenyl, C₂-C₁₀-haloalkenyl, C₂-C₁₀-alkynyl, or C₃-C₁₀-haloalkynyl;
R² is hydrogen, cyano, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₆-cycloalkyl, C₃-C₆-halocycloalkyl, C₁-C₁₀-alkoxy, or C₁-C₁₀-haloalkoxy;
R³ and R⁴ each independently are hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₁₀-cycloalkyl, C₃-C₁₀-halocycloalkyl, C₄-C₆-cycloalkenyl, C₄-C₆-halocycloalkenyl, or
R³ and R⁴ together can be C₂-C₁₀-alkylene, C₂-C₁₀-haloalkylene, C₄-C₁₀-alkenylene or C₄-C₁₀-haloalkenylene;
X is oxygen or sulfur;
R⁵ and R⁶ each independently are hydrogen, C₁-C₁₀-alkyl, which is unsubstituted or substituted with 1 to 3 substituents each independently selected from the group of cyano, nitro, hydroxy, NR⁷R⁸, C₁-C₁₀-alkoxy, C₁-C₁₀-haloalkoxy, C₁-C₁₀-alkylthio, C₁-C₁₀-haloalkylthio, C₃-C₆-cycloalkyl, C₃-C₆-halocycloalkyl and phenyl, wherein phenyl is unsubstituted or substituted with 1 to 5 substituents R^{a}; or
   C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkylsulfinyl, C₁-C₁₀-haloalkylsulfinyl, C₁-C₁₀-alkylsulfonyl, C₁-C₁₀-haloalkylsulfonyl, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁷R⁸N—C(═O)—, phenylcarbonyl, benzylcarbonyl or phenyl wherein phenylcarbonyl, benzylcarbonyl and phenyl are unsubstituted or substituted with 1 to 5 substituents R^{a}; or
   R⁵ and R⁶ each independently are a saturated, partially unsaturated or unsaturated 5- to 6-membered heterocyclic radical which may contain 1 to 4 heteroatoms selected from oxygen, nitrogen and sulfur and which radical may be bonded via a carbonyl (C(═O)) linkage, and which radical is unsubstituted or substituted with any combination of 1 to 4 groups R^{a};
      R^{a} is halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₁-C₁₀-alkylcarbonyl, C₁-C₁₀-haloalkylcarbonyl or R⁷R⁸N—C(═O)—;
      R⁷, R⁸ are each independently hydrogen, C₁-C₁₀-alkyl, C₁-C₁₀-haloalkyl, C₁-C₁₀-alkylcarbonyl, C₁-C₁₀-haloalkylcarbonyl, benzyl, phenyl, phenylcarbonyl, C₁-C₁₀-alkylsulfonyl, or C₁-C₁₀-haloalkylsulfonyl;
         or the enantiomers or diastereomers or salts thereof.

In addition, the present invention relates to processes and intermediates for preparing the compounds I, pesticidal compositions comprising compounds I, methods for the control of insects, acarids or nematodes by contacting the insect, acarid or nematode or their food supply, habitat or breeding grounds with a pesticidally effective amount of compounds or compositions of formula I.

Moreover, the present invention also relates to a method of protecting growing plants from attack or infestation by insects or acarids by applying to the foliage of the plants, or to the soil or water in which they are growing, with a pesticidally effective amount of compositions or compounds of formula I.

This invention also provides a method for treating, controlling, preventing or protecting animals against infestation or infection by parasites which comprises orally, topically or parenterally administering or applying to the animals a parasiticidally effective amount of compositions or compounds of formula I.

In spite of the commercial insecticides, acaricides and nematicides available today, damage to crops, both growing and harvested, caused by insects and nematodes still occurs. Therefore, there is a continuing need to develop new and more effective insecticidal, acaricidal and nematicidal agents.

It was therefore an object of the present invention to provide new pesticidal compositions, new compounds and new methods for the control of insects, acarids or nematodes and of protecting growing plants from attack or infestation by insects, arachnids or nematodes.

We have found that these objects are achieved by the compositions and the compounds of formula I. Furthermore, we have found processes and intermediates for preparing the compounds of formula I.

WO 04/020399 describes dicyano alkanoic acid ester compounds. The inventive dicyano alkanoic acid amides of formula I have not been described in the prior art.

Compounds of formula I are obtainable, for example, by a process wherein compound (II) is reacted with compound (III) to give compounds (I):

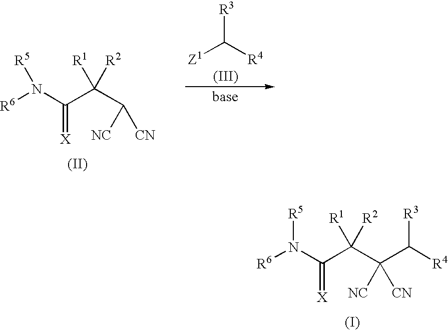

wherein X, R¹, R², R³, R⁴, R⁵ and R⁶ are as defined above for compounds of formula I and Z¹ represents a halogen atom, methanesulfonyl, trifluoromethanesulfonyl or toluenesulfonyl.

The reaction is generally carried out in the presence of a base in a solvent.

The solvent to be used in the reaction includes, for example, acid amides such as N,N-dimethylformamide, NMP (N-methylpyrrolidone) and the like, ethers such as diethyl ether, tetrahydrofuran and the like, sulfoxides and sulfones such as dimethylsulfoxide, sulfolane and the like, halogenated hydrocarbons such as 1,2-dichloroethane, chlorobenzene and the like, aromatic hydrocarbons such as toluene, xylene and the like, and mixtures thereof.

The base to be used in the reaction includes, for example, inorganic bases such as sodium hydride, sodium carbonate, potassium carbonate and the like, alkali metal alkoxides such as potassium t-butoxide and the like, alkali metal amides such as lithium diisopropylamide and the like, and organic bases such as dimethylaminopyridine, 1,4-diazabicyclo[2.2.2]octane, 1,8-diazabicyclo [5.4.0]-7-undecene and the like.

The amount of the base that can be used in the reaction is usually 1 to 10 moles relative to 1 mole of compound (II). In addition, additives such as crown ethers may be added to accelerate the reaction.

The amount of compound (III) to be used in the reaction is usually 1 to 10 moles, preferably 1 to 2 moles relative to 1 mole of compound (II).

The reaction temperature is usually in the range of −78° C. to 150° C., preferably in the range of −20° C. to 80° C. and the reaction time is usually in the range of 1 to 24 hours.

Some of the intermediates of formula II are novel. These are also subject of this invention.

After completion of the reaction, the compound of formula (I) can be isolated by employing conventional methods such as adding the reaction mixture to water, extracting with an organic solvent, concentrating the extract an the like. The isolated compound (I) can be purified by a technique such as chromatography, recrystallization and the like, if necessary.

The compound (II) can be prepared, for example, according to the route represented by the following scheme:

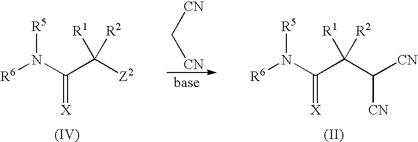

wherein X, R¹, R², R⁵ and R⁶ are as defined above for formula I and Z² represents a halogen atom, methanesulfonyl, trifluoromethanesulfonyl or toluenesulfonyl.

The reaction is generally carried out in the presence of a base in a solvent.

The solvent to be used in the reaction includes, for example, acid amides such as N,N-dimethylformamide, NMP and the like, ethers such as diethyl ether, tetrahydrofuran and the like, sulfoxides and sulfones such as dimethylsulfoxide (DMSO), sulfolane and the like, halogenated hydrocarbons such as 1,2-dichloroethane, chlorobenzene and the like, aromatic hydrocarbons such as toluene, xylene and the like, and mixtures thereof.

The base to be used in the reaction includes, for example, inorganic bases such as sodium hydride, sodium carbonate, potassium carbonate and the like, alkali metal alkoxides such as potassium t-butoxide and the like, alkali metal amides such as lithium diisopropylamide and the like, and organic bases such as dimethylaminopyridine, 1,4-diazabicyclo[2.2.2]octane, 1,8-diazabicyclo [5.4.0]-7-undecene and the like.

The amount of the base that can be used in the reaction is usually 1 to 10 moles relative to 1 mole of compound (IV). In addition, additives such as crown ethers may be added to accelerate the reaction.

The amount of malononitrile to be used in the reaction is usually 1 to 10 moles, preferably 1 to 2 moles relative to 1 mole of compound (IV).

The reaction temperature is usually in the range of −78° C. to 150° C., preferably in the range of −20° C. to 80° C. and the reaction time is usually in the range of 1 to 24 hours.

After completion of the reaction, the compound of formula (II) can be isolated by employing conventional methods such as adding the reaction mixture to water, extracting with an organic solvent, concentrating the extract and the like. The isolated compound (II) can be purified by a technique such as chromatography, recrystallization and the like, if necessary.

Compounds of formula IV can be obtained according to procedures known in the art, e.g. as described in J. March, Advanced Organic Chemistry, 4^{th} Edition, Wiley, 1992, p. 587-590.

According to a further embodiment of the present invention, compounds I can be obtained by a process wherein compound (V) is reacted with compound (IV) to give compounds (I):

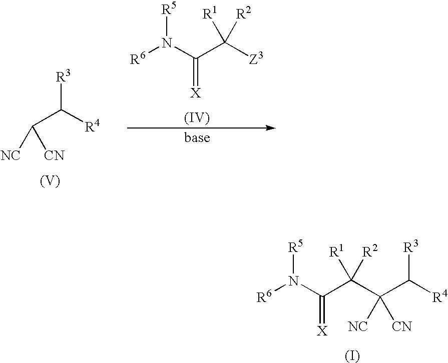

wherein, R¹, R², R³, R⁴, R⁵ and R⁶ are as defined above for compounds of formula I and Z³ represents a halogen atom, methanesulfonyl, trifluoromethanesulfonyl or toluenesulfonyl.

The reaction is generally carried out in the presence of base in a solvent.

The solvent to be used in the reaction includes, for example, acid amides such as N,N-dimethylformamide NMP and the like, ethers such as diethyl ether, tetrahydrofuran and the like, sulfoxides and sulfones such as dimethylsulfoxide, sulfolane and the like, halogenated hydrocarbons such as 1,2-dichloroethane, chlorobenzene and the like, aromatic hydrocarbons such as toluene, xylene and the like, and mixtures thereof.

The base to be used in the reaction includes, for example, inorganic bases such as sodium hydride, sodium carbonate, potassium carbonate and the like, alkali metal alkoxides such as potassium t-butoxide and the like, alkali metal amides such as lithium diisopropylamide and the like, and organic bases such as 4-dimethylaminopyridine, 1,4-diazabicyclo [2.2.2]octane, 1,8-diazabicyclo [5.4.0]-7-undecene and the like. The amount of the base that can be used in the reaction is usually 1 to 10 moles relative to 1 mole of compound (V).

The amount of compound (IV) to be used in the reaction is usually 1 to 10 moles, preferably 1 to 2 moles relative to 1 mole of compound (V).

The reaction temperature is usually in the range of −78° C. to 150° C., and the reaction time is usually in the range of 1 to 24 hours.

After completion of the reaction, the compound of formula (I) can be isolated by employing conventional methods such as adding the reaction mixture to water, extracting with an organic solvent, concentrating the extract and the like. The isolated compound (I) can be purified by a technique such as chromatography, recrystallization and the like, if necessary.

Compounds of formula (V) can be obtained according to procedures described in the art, e.g. in WO 02/89579, WO 02/90320, or WO 04/006677.

Compounds of formula (I) wherein X is sulfur may also be obtained from compounds of formula (I) wherein X is oxygen according the procedures known in the art such as described in J. March, Advanced Organic Chemistry, 4th Edition, Wiley, 1992, p. 893-895 and suitable modifications thereof.

The preparation of the compounds of formula I may lead to them being obtained as isomer mixtures. If desired, these can be resolved by the methods customary for this purpose, such as crystallization or chromatography, also on optically active adsorbate, to give the pure isomers.

The compounds of formula I may exist in different polymorphic forms which may exhibit different biological activity. These are also subject to this invention.

Agriculturally acceptable salts of the compounds I can be formed in a customary manner, e.g. by reaction with an acid of the anion in question.

In this specification and in the claims, reference will be made to a number of terms that shall be defined to have the following meanings:

“Salt” as used herein includes adducts of compounds I with maleic acid, dimaleic acid, fumaric acid, difumaric acid, methane sulfenic acid, methane sulfonic acid, and succinic acid. Moreover, included as “salts” are those that can form with, for example, amines, metals, alkaline earth metal bases or quaternary ammonium bases, including zwitterions. Suitable metal and alkaline earth metal hydroxides as salt formers include the salts of barium, aluminum, nickel, copper, manganese, cobalt zinc, iron, silver, lithium, sodium, potassium, magnesium or calcium. Additional salt formers include chloride, sulfate, acetate, carbonate, hydride, and hydroxide. Desirable salts include adducts of compounds I with maleic acid, dimaleic acid, fumaric acid, difumaric acid, and methane sulfonic acid.

“Halogen” will be taken to mean fluoro, chloro, bromo and iodo.

The term “alkyl” as used herein refers to a branched or unbranched saturated hydrocarbon group having 1 to 10 carbon atoms, such as C₁-C₆-alkyl, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

The term “haloalkyl” as used herein refers to a straight-chain or branched alkyl group having 1 to 10 carbon atoms (as mentioned above), where some or all of the hydrogen atoms in these groups may be replaced by halogen atoms as mentioned above, for example C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl;

Similarly, “alkoxy” and “alkylthio” refer to straight-chain or branched alkyl groups having 1 to 10 carbon atoms (as mentioned above) bonded through oxygen or sulfur linkages, respectively, at any bond in the alkyl group. Examples include methoxy, ethoxy, propoxy, isopropoxy, methylthio, ethylthio, propylthio, isopropylthio, and n-butylthio.

Similarly, “alkylsulfinyl” and “alkylsulfonyl” refer to straight-chain or branched alkyl groups having 1 to 10 carbon atoms (as mentioned above) bonded through —S(═O)— or —S(═O)₂-linkages, respectively, at any bond in the alkyl group. Examples include methylsulfinyl and methylsulfonyl.

The term “alkylcarbonyl” refers to straight-chain or branched alkyl groups having 1 to 10 carbon atoms (as mentioned above) bonded through a —C(═O)— linkage, respectively, at any bond in the alkyl group. Examples include acetyl and propionyl. The term “alkenyl” as used herein intends a branched or unbranched unsaturated hydrocarbon group having 2 to 6 carbon atoms and a double bond in any position, such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl; 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

The term “alkynyl” as used herein refers to a branched or unbranched unsaturated hydrocarbon group containing at least one triple bond, such as ethynyl, propynyl, 1-butynyl, 2-butynyl, and the like.

A saturated, partially unsaturated or unsaturated 5- to 6-membered heterocyclic radical which may contain 1 to 4 heteroatoms selected from oxygen, nitrogen and sulfur refers to e.g. a saturated, partially unsaturated or unsaturated 5-membered heterocyclic radical containing 1 to 4 nitrogen atoms, such as pyrrolidinyl, imidazolinyl, pyrazolyl, imidazolyl, triazolyl and tetrazolyl, or

a saturated, partially unsaturated or unsaturated 5-membered heterocyclic radical containing 0 to 3 nitrogen atoms and 1 sulfur or 1 oxygen atom, e.g. furanyl, thienyl, isoxazolinyl, isothiazolinyl, isoxazolyl, isothiazolyl, pyrazolyl, oxazolyl, thiazolyl, imidazolyl, oxadiazolyl and thiadiazolyl; or

a saturated, partially unsaturated or unsaturated 6-membered heterocyclic radical containing 1 to 3 nitrogen atoms e.g. piperidinyl, piperazinyl, pyridyl, pyridazinyl, pyrimidyl, pyrazinyl, and triazinyl; or

a saturated, partially unsaturated or unsaturated 6-membered heterocyclic radical containing 0 to 1 nitrogen atoms and 1 to 2 oxygen atoms or 1 to 2 sulfur atoms, e.g. dioxane, morpholine, dithiane, and thiomorpholine.

Preferred is a saturated, partially unsaturated or unsaturated 5-membered heterocyclic radical containing 2 to 3 nitrogen atoms, such as pyrazolyl, imidazolyl and triazolyl; or a saturated, partially unsaturated or unsaturated 5-membered heterocyclic radical containing 1 or 2 nitrogen atoms and 1 oxygen atom, e.g. isoxazolyl, oxazolyl, oxadiazolyl, thiadzoly and thiadiazolyl; or

a saturated, partially unsaturated or unsaturated 6-membered heterocyclic radical containing 1 to 2 nitrogen atoms e.g. pyridine, pyridazine, pyrimidine and pyrazine; or a saturated, partially unsaturated or unsaturated 6-membered heterocyclic radical containing 0 to 1 nitrogen atoms and 1 to 2 oxygen atoms or 1 to 2 sulfur atoms, e.g. dioxane, morpholine, dithiane, and thiomorpholine.

Cycloalkyl: monocyclic 3- to 6-, 8-, or 10-membered saturated carbon atom rings, e.g. C₃-C₁₀-cycloalkyl such as cyclopropyl, cyclobutyl, cyclopentyl, cyclohexyl, cycloheptyl, cyclooctyl, cyclononyl, or cyclodecyl.

With respect to the intended use of the compounds of formula I, particular preference is given to the following meanings of the substituents, in each case on their own or in combination:

A compound of formula I wherein X is oxygen.

A compound of formula I wherein R¹ is hydrogen and R² is methyl.

A compound of formula I wherein R² is hydrogen, methyl or cyano.

A compound of formula I wherein R¹ and R² are hydrogen.

A compound of formula I wherein R¹ and R² are methyl.

A compound of formula I wherein R⁴ is hydrogen, C₂-C₆-alkenyl, such as vinyl, 2-propenyl, or 2-methyl-1-propenyl, C₂-C₆-haloalkenyl, such as 2,2-difluorovinyl, 1-(trifluoromethyl)vinyl, 3,3-difluoro-2-propenyl, 2,3,3-trifluoro-2-propenyl, or 3,3,3-trifluoro-1-propenyl, C₂-C₆-haloalkynyl, C₁-C₆-fluoroalkyl, such as fluoromethyl, trifluoromethyl, 2,2-difluoroethyl, 2,2,2-trifluoroethyl, pentafluoroethyl, 3,3,3-trifluoropropyl, or 2,2,3,3,3-pentafluoropropyl, C₃-C₆-cycloalkyl, 2,2-dichlorocyclopropyl, cyclopropyl, or cyclobutyl.

A compound of formula I wherein R³ is hydrogen.

A compound of formula I wherein R³ is hydrogen and R⁴ is a C₂-C₆-alkenyl. A compound of formula I wherein R³ is hydrogen and R⁴ is vinyl, 2-propenyl or 2-methyl-1-propenyl.

A compound of formula I wherein R³ is hydrogen and R⁴ is a C₂-C₆-haloalkenyl.

A compound of formula I wherein R³ is hydrogen and R⁴ is 2,2-difluorovinyl, 1-(trifluoromethyl)vinyl, 3,3-difluoro-2-propenyl, 2,3,3-trifluoro-2-propenyl or 3,3,3-trifluoro-1-propenyl.

A compound of formula I wherein R³ is hydrogen and R⁴ is a C₁-C₆-haloalkyl.

A compound of formula I wherein R³ is hydrogen and R⁴ is fluoromethyl, 2,2-difluoroethyl, 2,2,2-trifluoroethyl, 1,1,2,2,2-pentafluoroethyl, 3,3,3-trifluoropropyl or 2,2,3,3,3-pentafluoropropyl.

A compound of formula I wherein R³ is hydrogen and R⁴ is a C₃-C₆-cycloalkyl or C₃-C₆-halocycloalkyl

A compound of formula I wherein R³ is hydrogen and R⁴ is cyclopropyl, cyclobutyl or 2,2-dichlorocyclopropyl.

A compound of formula I wherein R¹, R² and R³ are hydrogen and R⁴ is vinyl, 2-propenyl or 2-methyl-2-propenyl.

A compound of formula I wherein R¹, R² and R³ are hydrogen and R⁴ is 2,2-difluorovinyl, 1-(trifluoromethyl)vinyl, 3,3-difluoro-2-propenyl, 2,3,3-trifluoro-2-propenyl or 3,3,3-trifluoro-1-propenyl.

A compound of formula I wherein R¹ and R² are methyl, R³ is hydrogen and R⁴ is 2,2-difluorovinyl, 1-(trifluoromethyl)vinyl, 3,3-difluoro-2-propenyl, 2,3,3-trifluoro-2-propenyl or 3,3,3-trifluoro-1-propenyl.

A compound of formula I wherein R¹ and R³ are hydrogen, R² is methyl and R⁴ is 2,2-difluorovinyl, 1-(trifluoromethyl)vinyl, 3,3-difluoro-2-propenyl, 2,3,3-trifluoro-2-propenyl or 3,3,3-trifluoro-1-propenyl.

A compound of formula I wherein R¹, R² and R³ are hydrogen, R⁴ is fluoromethyl, 2,2-difluoroethyl, 2,2,2-trifluoroethyl, pentafluoroethyl, 3,3,3-trifluoropropyl or 2,2,3,3,3-pentafluoropropyl.

A compound of formula I wherein R¹ and R³ are hydrogen, R² is methyl and R⁴ is fluoromethyl, 2,2-difluoroethyl, 2,2,2-trifluoroethyl, pentafluoroethyl, 3,3,3-trifluoropropyl or 2,2,3,3,3-pentafluoropropyl.

A compound of formula I wherein R¹ and R² are methyl, R³ is hydrogen, and R⁴ is fluoromethyl, 2,2-difluoroethyl, 2,2,2-trifluoroethyl, pentafluoroethyl, 3,3,3-trifluoropropyl or 2,2,3,3,3-pentafluoropropyl.

A compound of formula I wherein R¹, R² and R³ are hydrogen and R⁴ is cyclopropyl, cyclobutyl or 2,2-dichlorocyclopropyl.

A compound of formula I wherein R⁵ and R⁶ are each independently hydrogen, C₁-C₁₀-alkyl, which is unsubstituted or substituted by 1 substituent selected from the group of cyano, nitro, C₁-C₁₀-alkoxy, C₁-C₁₀-alkylthio, phenyl and C₃-C₆-cycloalkyl; or C₁-C₁₀-haloalkyl, C₃-C₁₀-cycloalkyl, C₂-C₁₀-alkenyl, C₂-C₁₀-alkynyl, C₁-C₁₀-alkylcarbonyl, C₁-C₁₀-haloalkylcarbonyl, phenylcarbonyl, benzylcarbonyl or phenyl.

A compound of formula I wherein R⁵ and R⁶ are each independently hydrogen, C₁-C₅-alkyl, which is unsubstituted or substituted by 1 substituent selected from the group cyano, C₁-C₄-alkoxy, phenyl and C₃-C₆-cycloalkyl; or C₁-C₅-haloalkyl, C₃-C₆-cylcoalkyl, C₂-C₅-alkynyl, C₁-C₅-alkylcarbonyl, phenylcarbonyl or phenyl.

A compound of formula I wherein R^{a} is halogen, cyano, C₁-C₄-alkyl, C₁-C₄-haloalkyl, C₁-C₄-alkoxy, C₁-C₄-haloalkoxy, C₁-C₄-alkylthio or C₁-C₄-haloalkylthio.

A compound of formula I wherein the substituents phenyl, phenylcarbonyl, benzylcarbonyl or the 5- to 6-membered heterocyclic radical, all as defined for the groups R⁵ and R⁶, are unsubstituted.

Especially preferred are compounds of formula I wherein R¹, R² and R³ are hydrogen.

Besides, especially preferred are compounds of formula I wherein R⁵ and R⁶ are each independently selected from the group hydrogen, C₁-C₄-alkyl, C₁-C₄-haloalkyl, cyano-C₁-C₄-alkyl, C₁-C₄-alkoxy-C₁-C₄-alkyl, C₁-C₄-haloalkoxy-C₁-C₄-alkyl, phenyl, phenylmethyl, phenylcarbonyl, C₁-C₄-alkylcarbonyl, C₁-C₄-alkylsulfonyl, phenylsulfonyl, and C₁-C₄-alkylphenyl-sulfonyl.

Even more preferred are compounds of formula I wherein R⁵ and R⁶ are each independently selected from the group hydrogen, methyl, ethyl, propyl, isopropyl, cyclopropyl, n-butyl, iso-butyl, tert.-butyl, cyanomethyl, trifluormethyl, CF(CF₃)₃, methoxymethyl, fluoromethoxymethyl, allyl, propargyl, phenyl, phenylmethyl, phenylcarbonyl, methylcarbonyl, phenylsulfonyl, and para-methylphenylsulfonyl.

With respect to their use, particular preference is given to the compounds IA compiled in the tables below. Moreover, the groups mentioned for a substituent in the tables are on their own, independently of the combination in which they are mentioned, a particularly preferred embodiment of the substituent in question.

Table 1

Compounds of the formula IA wherein R⁴ denotes CH═CH₂ and the combination of R⁵ and R⁶ in each case corresponds to a row of Table A.

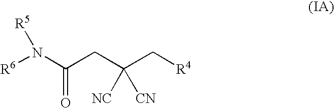

Table 2

Compounds of the formula IA wherein R⁴ denotes CH₂—CH═CH₂ and the combination of R⁵ and R⁶ in each case corresponds to a row of Table A.

Table 3

Compounds of the formula IA wherein R⁴ denotes CH₂CF₃ and the combination of R⁵ and R⁶ in each case corresponds to a row of Table A.

Table 4

Compounds of the formula IA wherein R⁴ denotes cyclopropyl and the combination of R⁵ and R⁶ in each case corresponds to a row of Table A.

Table 5

Compounds of the formula IA wherein R⁴ denotes cyclobutyl and the combination of R⁵ and R⁶ in each case corresponds to a row of Table A.

Table 6

Compounds of the formula IA wherein R⁴ denotes 1-methyl-cyclopropyl and the combination of R⁵ and R⁶ in each case corresponds to a row of Table A.

Table 7

Compounds of the formula IA wherein R⁴ denotes CF₃ and the combination of R⁵ and R⁶ in each case corresponds to a row of Table A.

TABLE A

| No. | R⁵ | R⁶ |
|---|---|---|
| IA-1 | H | H |
| IA-2 | CH₃ | H |
| IA-3 | CH₂CH₃ | H |
| IA-4 | CH₂CH₂CH₃ | H |
| IA-5 | CH(CH₃)₂ | H |
| IA-6 | Cyclo-C₃H₅ | H |
| IA-7 | CH₂CH₂CH₂CH₃ | H |
| IA-8 | CH₂CH(CH₃)₂ | H |
| IA-9 | C(CH₃)₃ | H |
| IA-10 | CH₂CHCH₂ | H |
| IA-11 | CH₂CCH | H |
| IA-12 | C₆H₅ | H |
| IA-13 | CH₂C₆H₅ | H |
| IA-14 | CH₂CN | H |
| IA-15 | CF₃ | H |
| IA-16 | CH₂F | H |
| IA-17 | CF(CF₃)₂ | H |
| IA-18 | CH₂OCH₃ | H |
| IA-19 | CH₂OCH₂F | H |
| IA-20 | C(═O)CH₃ | H |
| IA-21 | C(═O)C₆H₅ | H |
| IA-22 | S(═O)₂C₆H₅ | H |
| IA-23 | S(═O)₂[(p-CH₃)C₆H₄] | H |
| IA-24 | CH₃ | CH₃ |
| IA-25 | CH₂CH₃ | CH₃ |
| IA-26 | CH₂CH₂CH₃ | CH₃ |
| IA-27 | CH(CH₃)₂ | CH₃ |
| IA-28 | Cyclo-C₃H₅ | CH₃ |
| IA-29 | CH₂CH₂CH₂CH₃ | CH₃ |
| IA-30 | CH₂CH(CH₃)₂ | CH₃ |
| IA-31 | C(CH₃)₃ | CH₃ |
| IA-32 | CH₂CHCH₂ | CH₃ |
| IA-33 | CH₂CCH | CH₃ |
| IA-34 | C₆H₅ | CH₃ |
| IA-35 | CH₂C₆H₅ | CH₃ |
| IA-36 | CH₂CN | CH₃ |
| IA-37 | CF₃ | CH₃ |
| IA-38 | CH₂F | CH₃ |
| IA-39 | CF(CF₃)₂ | CH₃ |
| IA-40 | CH₂OCH₃ | CH₃ |
| IA-41 | CH₂OCH₂F | CH₃ |
| IA-42 | C(═O)CH₃ | CH₃ |
| IA-43 | C(═O)C₆H₅ | CH₃ |
| IA-44 | S(═O)₂C₆H₅ | CH₃ |
| IA-45 | S(═O)₂[(p-CH₃)C₆H₄] | CH₃ |
| IA-46 | CH₂CH₃ | CH₂CH₃ |
| IA-47 | CH₂CH₂CH₃ | CH₂CH₃ |
| IA-48 | CH(CH₃)₂ | CH₂CH₃ |
| IA-49 | Cyclo-C₃H₅ | CH₂CH₃ |
| IA-50 | CH₂CH₂CH₂CH₃ | CH₂CH₃ |
| IA-51 | CH₂CH(CH₃)₂ | CH₂CH₃ |
| IA-52 | C(CH₃)₃ | CH₂CH₃ |
| IA-53 | CH₂CHCH₂ | CH₂CH₃ |
| IA-54 | CH₂CCH | CH₂CH₃ |
| IA-55 | C₆H₅ | CH₂CH₃ |
| IA-56 | CH₂C₆H₅ | CH₂CH₃ |
| IA-57 | CH₂CN | CH₂CH₃ |
| IA-58 | CF₃ | CH₂CH₃ |
| IA-59 | CH₂F | CH₂CH₃ |
| IA-60 | CF(CF₃)₂ | CH₂CH₃ |
| IA-61 | CH₂OCH₃ | CH₂CH₃ |
| IA-62 | CH₂OCH₂F | CH₂CH₃ |
| IA-63 | C(═O)CH₃ | CH₂CH₃ |
| IA-64 | C(═O)C₆H₅ | CH₂CH₃ |
| IA-65 | S(═O)₂C₆H₅ | CH₂CH₃ |
| IA-66 | S(═O)₂[(p-CH₃)C₆H₄] | CH₂CH₃ |
| IA-67 | CH₂CH₂CH₃ | CH₂CH₂CH₃ |
| IA-68 | CH(CH₃)₂ | CH₂CH₂CH₃ |
| IA-69 | Cyclo-C₃H₅ | CH₂CH₂CH₃ |
| IA-70 | CH₂CH₂CH₂CH₃ | CH₂CH₂CH₃ |
| IA-71 | CH₂CH(CH₃)₂ | CH₂CH₂CH₃ |
| IA-72 | C(CH₃)₃ | CH₂CH₂CH₃ |
| IA-73 | CH₂CHCH₂ | CH₂CH₂CH₃ |
| IA-74 | CH₂CCH | CH₂CH₂CH₃ |
| IA-75 | C₆H₅ | CH₂CH₂CH₃ |
| IA-76 | CH₂C₆H₅ | CH₂CH₂CH₃ |
| IA-77 | CH₂CN | CH₂CH₂CH₃ |
| IA-78 | CF₃ | CH₂CH₂CH₃ |
| IA-79 | CH₂F | CH₂CH₂CH₃ |
| IA-80 | CF(CF₃)₂ | CH₂CH₂CH₃ |
| IA-81 | CH₂OCH₃ | CH₂CH₂CH₃ |
| IA-82 | CH₂OCH₂F | CH₂CH₂CH₃ |
| IA-83 | C(═O)CH₃ | CH₂CH₂CH₃ |
| IA-84 | C(═O)C₆H₅ | CH₂CH₂CH₃ |
| IA-85 | S(═O)₂C₆H₅ | CH₂CH₂CH₃ |
| IA-86 | S(═O)₂[(p-CH₃)C₆H₄] | CH₂CH₂CH₃ |
| IA-87 | CH(CH₃)₂ | CH(CH₃)₂ |
| IA-88 | Cyclo-C₃H₅ | CH(CH₃)₂ |
| IA-89 | CH₂CH₂CH₂CH₃ | CH(CH₃)₂ |
| IA-90 | CH₂CH(CH₃)₂ | CH(CH₃)₂ |
| IA-91 | C(CH₃)₃ | CH(CH₃)₂ |
| IA-92 | CH₂CHCH₂ | CH(CH₃)₂ |
| IA-93 | CH₂CCH | CH(CH₃)₂ |
| IA-94 | C₆H₅ | CH(CH₃)₂ |
| IA-95 | CH₂C₆H₅ | CH(CH₃)₂ |
| IA-96 | CH₂CN | CH(CH₃)₂ |
| IA-97 | CF₃ | CH(CH₃)₂ |
| IA-98 | CH₂F | CH(CH₃)₂ |
| IA-99 | CF(CF₃)₂ | CH(CH₃)₂ |
| IA-100 | CH₂OCH₃ | CH(CH₃)₂ |
| IA-101 | CH₂OCH₂F | CH(CH₃)₂ |
| IA-102 | C(═O)CH₃ | CH(CH₃)₂ |
| IA-103 | C(═O)C₆H₅ | CH(CH₃)₂ |
| IA-104 | S(═O)₂C₆H₅ | CH(CH₃)₂ |
| IA-105 | S(═O)₂[(p-CH₃)C₆H₄] | CH(CH₃)₂ |
| IA-106 | Cyclo-C₃H₅ | Cyclo-C₃H₅ |
| IA-107 | CH₂CH₂CH₂CH₃ | Cyclo-C₃H₅ |
| IA-108 | CH₂CH(CH₃)₂ | Cyclo-C₃H₅ |
| IA-109 | C(CH₃)₃ | Cyclo-C₃H₅ |
| IA-110 | CH₂CHCH₂ | Cyclo-C₃H₅ |
| IA-111 | CH₂CCH | Cyclo-C₃H₅ |
| IA-112 | C₆H₅ | Cyclo-C₃H₅ |
| IA-113 | CH₂C₆H₅ | Cyclo-C₃H₅ |
| IA-114 | CH₂CN | Cyclo-C₃H₅ |
| IA-115 | CF₃ | Cyclo-C₃H₅ |
| IA-116 | CH₂F | Cyclo-C₃H₅ |
| IA-117 | CF(CF₃)₂ | Cyclo-C₃H₅ |
| IA-118 | CH₂OCH₃ | Cyclo-C₃H₅ |
| IA-119 | CH₂OCH₂F | Cyclo-C₃H₅ |
| IA-120 | C(═O)CH₃ | Cyclo-C₃H₅ |
| IA-121 | C(═O)C₆H₅ | Cyclo-C₃H₅ |
| IA-122 | S(═O)₂C₆H₅ | Cyclo-C₃H₅ |
| IA-123 | S(═O)₂[(p-CH₃)C₆H₄] | Cyclo-C₃H₅ |
| IA-124 | CH₂CH₂CH₂CH₃ | CH₂CH₂CH₂CH₃ |
| IA-125 | CH₂CH(CH₃)₂ | CH₂CH₂CH₂CH₃ |
| IA-126 | C(CH₃)₃ | CH₂CH₂CH₂CH₃ |
| IA-127 | CH₂CHCH₂ | CH₂CH₂CH₂CH₃ |
| IA-128 | CH₂CCH | CH₂CH₂CH₂CH₃ |
| IA-129 | C₆H₅ | CH₂CH₂CH₂CH₃ |
| IA-130 | CH₂C₆H₅ | CH₂CH₂CH₂CH₃ |
| IA-131 | CH₂CN | CH₂CH₂CH₂CH₃ |
| IA-132 | CF₃ | CH₂CH₂CH₂CH₃ |
| IA-133 | CH₂F | CH₂CH₂CH₂CH₃ |
| IA-134 | CF(CF₃)₂ | CH₂CH₂CH₂CH₃ |
| IA-135 | CH₂OCH₃ | CH₂CH₂CH₂CH₃ |
| IA-136 | CH₂OCH₂F | CH₂CH₂CH₂CH₃ |
| IA-137 | C(═O)CH₃ | CH₂CH₂CH₂CH₃ |
| IA-138 | C(═O)C₆H₅ | CH₂CH₂CH₂CH₃ |
| IA-139 | S(═O)₂C₆H₅ | CH₂CH₂CH₂CH₃ |
| IA-140 | S(═O)₂[(p-CH₃)C₆H₄] | CH₂CH₂CH₂CH₃ |
| IA-141 | CH₂CH(CH₃)₂ | CH₂CH(CH₃)₂ |
| IA-142 | C(CH₃)₃ | CH₂CH(CH₃)₂ |
| IA-143 | CH₂CHCH₂ | CH₂CH(CH₃)₂ |
| IA-144 | CH₂CCH | CH₂CH(CH₃)₂ |
| IA-145 | C₆H₅ | CH₂CH(CH₃)₂ |
| IA-146 | CH₂C₆H₅ | CH₂CH(CH₃)₂ |
| IA-147 | CH₂CN | CH₂CH(CH₃)₂ |
| IA-148 | CF₃ | CH₂CH(CH₃)₂ |
| IA-149 | CH₂F | CH₂CH(CH₃)₂ |
| IA-150 | CF(CF₃)₂ | CH₂CH(CH₃)₂ |
| IA-151 | CH₂OCH₃ | CH₂CH(CH₃)₂ |
| IA-152 | CH₂OCH₂F | CH₂CH(CH₃)₂ |
| IA-153 | C(═O)CH₃ | CH₂CH(CH₃)₂ |
| IA-154 | C(═O)C₆H₅ | CH₂CH(CH₃)₂ |
| IA-155 | S(═O)₂C₆H₅ | CH₂CH(CH₃)₂ |
| IA-156 | S(═O)₂[(p-CH₃)C₆H₄] | CH₂CH(CH₃)₂ |
| IA-157 | C(CH₃)₃ | C(CH₃)₃ |
| IA-158 | CH₂CHCH₂ | C(CH₃)₃ |
| IA-159 | CH₂CCH | C(CH₃)₃ |
| IA-160 | C₆H₅ | C(CH₃)₃ |
| IA-161 | CH₂C₆H₅ | C(CH₃)₃ |
| IA-162 | CH₂CN | C(CH₃)₃ |
| IA-163 | CF₃ | C(CH₃)₃ |
| IA-164 | CH₂F | C(CH₃)₃ |
| IA-165 | CF(CF₃)₂ | C(CH₃)₃ |
| IA-166 | CH₂OCH₃ | C(CH₃)₃ |
| IA-167 | CH₂OCH₂F | C(CH₃)₃ |
| IA-168 | C(═O)CH₃ | C(CH₃)₃ |
| IA-169 | C(═O)C₆H₅ | C(CH₃)₃ |
| IA-170 | S(═O)₂C₆H₅ | C(CH₃)₃ |
| IA-171 | S(═O)₂[(p-CH₃)C₆H₄] | C(CH₃)₃ |
| IA-172 | CH₂CHCH₂ | CH₂CHCH₂ |
| IA-173 | CH₂CCH | CH₂CHCH₂ |
| IA-174 | C₆H₅ | CH₂CHCH₂ |
| IA-175 | CH₂C₆H₅ | CH₂CHCH₂ |
| IA-176 | CH₂CN | CH₂CHCH₂ |
| IA-177 | CF₃ | CH₂CHCH₂ |
| IA-178 | CH₂F | CH₂CHCH₂ |
| IA-179 | CF(CF₃)₂ | CH₂CHCH₂ |
| IA-180 | CH₂OCH₃ | CH₂CHCH₂ |
| IA-181 | CH₂OCH₂F | CH₂CHCH₂ |
| IA-182 | C(═O)CH₃ | CH₂CHCH₂ |
| IA-183 | C(═O)C₆H₅ | CH₂CHCH₂ |
| IA-184 | S(═O)₂C₆H₅ | CH₂CHCH₂ |
| IA-185 | S(═O)₂[(p-CH₃)C₆H₄] | CH₂CHCH₂ |
| IA-186 | CH₂CCH | CH₂CCH |
| IA-187 | C₆H₅ | CH₂CCH |
| IA-188 | CH₂C₆H₅ | CH₂CCH |
| IA-189 | CH₂CN | CH₂CCH |
| IA-190 | CF₃ | CH₂CCH |
| IA-191 | CH₂F | CH₂CCH |
| IA-192 | CF(CF₃)₂ | CH₂CCH |
| IA-193 | CH₂OCH₃ | CH₂CCH |
| IA-194 | CH₂OCH₂F | CH₂CCH |
| IA-195 | C(═O)CH₃ | CH₂CCH |
| IA-196 | C(═O)C₆H₅ | CH₂CCH |
| IA-197 | S(═O)₂C₆H₅ | CH₂CCH |
| IA-198 | S(═O)₂[(p-CH₃)C₆H₄] | CH₂CCH |
| IA-199 | C₆H₅ | C₆H₅ |
| IA-200 | CH₂C₆H₅ | C₆H₅ |
| IA-201 | CH₂CN | C₆H₅ |
| IA-202 | CF₃ | C₆H₅ |
| IA-203 | CH₂F | C₆H₅ |
| IA-204 | CF(CF₃)₂ | C₆H₅ |
| IA-205 | CH₂OCH₃ | C₆H₅ |
| IA-206 | CH₂OCH₂F | C₆H₅ |
| IA-207 | C(═O)CH₃ | C₆H₅ |
| IA-208 | C(═O)C₆H₅ | C₆H₅ |
| IA-209 | S(═O)₂C₆H₅ | C₆H₅ |
| IA-210 | S(═O)₂[(p-CH₃)C₆H₄] | C₆H₅ |
| IA-211 | CH₂C₆H₅ | CH₂C₆H₅ |
| IA-212 | CH₂CN | CH₂C₆H₅ |
| IA-213 | CF₃ | CH₂C₆H₅ |
| IA-214 | CH₂F | CH₂C₆H₅ |
| IA-215 | CF(CF₃)₂ | CH₂C₆H₅ |
| IA-216 | CH₂OCH₃ | CH₂C₆H₅ |
| IA-217 | CH₂OCH₂F | CH₂C₆H₅ |
| IA-218 | C(═O)CH₃ | CH₂C₆H₅ |
| IA-219 | C(═O)C₆H₅ | CH₂C₆H₅ |
| IA-220 | S(═O)₂C₆H₅ | CH₂C₆H₅ |
| IA-221 | S(═O)₂[(p-CH₃)C₆H₄] | CH₂C₆H₅ |
| IA-222 | CH₂CN | CH₂CN |
| IA-223 | CF₃ | CH₂CN |
| IA-224 | CH₂F | CH₂CN |
| IA-225 | CF(CF₃)₂ | CH₂CN |
| IA-226 | CH₂OCH₃ | CH₂CN |
| IA-227 | CH₂OCH₂F | CH₂CN |
| IA-228 | C(═O)CH₃ | CH₂CN |
| IA-229 | C(═O)C₆H₅ | CH₂CN |
| IA-230 | S(═)₂C₆H₅ | CH₂CN |
| IA-231 | S(═O)₂[(p-CH₃)C₆H₄] | CH₂CN |
| IA-232 | CF₃ | CF₃ |
| IA-233 | CH₂F | CF₃ |
| IA-234 | CF(CF₃)₂ | CF₃ |
| IA-235 | CH₂OCH₃ | CF₃ |
| IA-236 | CH₂OCH₂F | CF₃ |
| IA-237 | C(═O)CH₃ | CF₃ |
| IA-238 | C(═O)C₆H₅ | CF₃ |
| IA-239 | S(═)₂C₆H₅ | CF₃ |
| IA-240 | S(═O)₂[(p-CH₃)C₆H₄] | CF₃ |
| IA-241 | CH₂F | CH₂F |
| IA-242 | CF(CF₃)₂ | CH₂F |
| IA-243 | CH₂OCH₃ | CH₂F |
| IA-244 | CH₂OCH₂F | CH₂F |
| IA-245 | C(═O)CH₃ | CH₂F |
| IA-246 | C(═O)C₆H₅ | CH₂F |
| IA-247 | S(═)₂C₆H₅ | CH₂F |
| IA-248 | S(═O)₂[(p-CH₃)C₆H₄] | CH₂F |
| IA-249 | CF(CF₃)₂ | CF(CF₃)₂ |
| IA-250 | CH₂OCH₃ | CF(CF₃)₂ |
| IA-251 | CH₂OCH₂F | CF(CF₃)₂ |
| IA-252 | C(═O)CH₃ | CF(CF₃)₂ |
| IA-253 | C(═O)C₆H₅ | CF(CF₃)₂ |
| IA-254 | S(═)₂C₆H₅ | CF(CF₃)₂ |
| IA-255 | S(═O)₂[(p-CH₃)C₆H₄] | CF(CF₃)₂ |
| IA-256 | CH₂OCH₃ | CH₂OCH₃ |
| IA-257 | CH₂OCH₂F | CH₂OCH₃ |
| IA-258 | C(═O)CH₃ | CH₂OCH₃ |
| IA-259 | C(═O)C₆H₅ | CH₂OCH₃ |
| IA-260 | S(═)₂C₆H₅ | CH₂OCH₃ |
| IA-261 | S(═O)₂[(p-CH₃)C₆H₄] | CH₂OCH₃ |
| IA-262 | CH₂OCH₂F | CH₂OCH₂F |
| IA-263 | C(═O)CH₃ | CH₂OCH₂F |
| IA-264 | C(═O)C₆H₅ | CH₂OCH₂F |
| IA-265 | S(═)₂C₆H₅ | CH₂OCH₂F |
| IA-266 | S(═O)₂[(p-CH₃)C₆H₄] | CH₂OCH₂F |

In the intermediates of formula II, the substituents X, R¹, R², R⁵ and R⁶ have the same preferred meanings as for compound 1.

The compounds of the formula I are especially suitable for efficiently combating the following pests:

insects from the order of the lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Chematobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix viridana, Trichoplusia ni and Zeiraphera canadensis,

beetles (Coleoptera), for example Agrilus sinuatus, Agriotes lineatus, Agriotes obscurus, Amphimallus solstitialis, Anisandrus dispar, Anthonomus grandis, Anthonomus pomorum, Aphthona euphoridae, Athous haemorrhoidalis, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Cetonia aurata, Ceuthorrhynchus assimilis, Ceuthorrhynchus napi, Chaetocnema tibialis, Conoderus vespertinus, Crioceris asparagi, Ctenicera ssp., Diabrotica longicornis, Diabrotica semipunctata, Diabrotica 12-punctata Diabrotica speciosa, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Otiorrhynchus ovatus, Phaedon cochleariae, Phyllobius pyri, Phyllotreta chrysocephala, Phyllophaga sp., Phyllopertha horticola, Phyllotreta nemorum, Phyllotreta striolata, Popillia japonica, Sitona lineatus and Sitophilus granaria,

flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliomyia hominivorax, Contarinia sorghicola Cordylobia anthropophaga, Culicoides furens, Culex pipiens, Culex nigripalpus, Culex quinquefasciatus, Culex tarsalis, Culiseta inornata, Culiseta melanura, Dacus cucurbitae, Dacus oleae, Dasineura brassicae, Delia antique, Delia coarctata, Delia platura, Delia radicum, Dermatobia hominis, Fannia canicularis, Geomyza Tripunctata, Gasterophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza trifolii, Lucilia caprina, Lucilia cuprina, Lucilia sericata, Lycoria pectoralis, Mansonia titillanus, Mayetiola destructor, Musca domestica, Muscina stabulans, Oestrus ovis, Opomyza florum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosimulium mixtum, Rhagoletis cerasi, Rhagoletis pomonella, Sarcophaga haemorrhoidalis, Sarcophaga sp., Simulium vittatum, Stomoxys calcitrans, Tabanus bovinus, Tabanus atratus, Tabanus lineola, and Tabanus similis, Tipula oleracea, and Tipula paludosa

thrips (Thysanoptera), e.g. Dichromothrips corbetti, Dichromothrips ssp, Frankliniella fusca, Frankliniella occidentalis, Frankliniella tritici, Scirtothrips citri, Thrips oryzae, Thrips palmi and Thrips tabaci,

termites (Isoptera), e.g. Calotermes flavicollis, Leucotermes flavipes, Heterotermes aureus, Reticulitermes flavipes, Reticulitermes virginicus, Reticulitermes lucifugus, Termes natalensis, and Coptotermes formosanus,

cockroaches (Blattaria—Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,

true bugs (Hemiptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis, Thyanta perditor, Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisia argentifolii, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzus persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, Viteus vitifolii, Cimex lectularius, Cimex hemipterus, Reduvius senilis, Triatoma spp., and Arilus critatus.

ants, bees, wasps, sawflies (Hymenoptera), e.g. Athalia rosae, Atta cephalotes, Atta capiguara, Atta cephalotes, Atta laevigata, Atta robusta, Atta sexdens, Atta texana, Crematogaster spp., Hoplocampa minuta, Hoplocampa testudinea, Monomorium pharaonis, Solenopsis geminata, Solenopsis invicta, Solenopsis richteri, Solenopsis xyloni, Pogonomyrmex barbatus, Pogonomyrmex californicus, Pheidole megacephala, Dasymutilla occidentalis, Bombus spp. Vespula squamosa, Paravespula vulgaris, Paravespula pennsylvanica, Paravespula germanica, Dolichovespula maculata, Vespa crabro, Polistes rubiginosa, Camponotus floridanus, and Linepithema humile,

crickets, grasshoppers, locusts (Orthoptera), e.g. Acheta domestica, Gryllotalpa gryllotalpa, Locusta migratoria, Melanoplus bivittatus, Melanoplus femurrubrum, Melanoplus mexicanus, Melanoplus sanguinipes, Melanoplus spretus, Nomadacris septemfasciata, Schistocerca americana, Schistocerca gregaria, Dociostaurus maroccanus, Tachycines asynamorus, Oedaleus senegalensis, Zonozerus variegatus, Hieroglyphus daganensis, Kraussaria angulifera, Calliptamus italicus, Chortoicetes terminifera, and Locustana pardalina,

Arachnoidea, such as arachnids (Acarina), e.g. of the families Argasidae, Ixodidae and Sarcoptidae, such as Amblyomma americanum, Amblyomma variegatum, Ambryomma maculatum, Argas persicus, Boophilus annulatus, Boophilus decoloratus, Boophilus microplus, Dermacentor silvarum, Dermacentor andersoni, Dermacentor variabilis, Hyalomma truncatum, Ixodes ricinus, Ixodes rubicundus, Ixodes scapularis, Ixodes holocyclus, Ixodes pacificus, Ornithodorus moubata, Ornithodorus hermsi, Ornithodorus turicata, Ornithonyssus bacoti, Otobius megnini, Dermanyssus gallinae, Psoroptes ovis, Rhipicephalus sanguineus, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and Oligonychus pratensis; Araneida, e.g. Latrodectus mactans, and Loxosceles reclusa,

fleas (Siphonaptera), e.g. Ctenocephalides felis, Ctenocephalides canis, Xenopsylla cheopis, Pulex irritans, Tunga penetrans, and Nosopsyllus fasciatus,

silverfish, firebrat (Thysanura), e.g. Lepisma saccharina and Thermobia domestica,

centipedes (Chilopoda), e.g. Scutigera coleoptrata,

millipedes (Diplopoda), e.g. Narceus spp.,

Earwigs (Dermaptera), e.g. forficula auricularia,

lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus,

Plant parasitic nematodes such as root-knot nematodes, Meloidogyne arenaria, Meloidogyne chitwoodi, Meloidogyne exigua, Meloidogyne hapla, Meloidogyne incognita, Meloidogyne javanica and other Meloidogyne species; cyst nematodes, Globodera rostochiensis, Globodera pallida, Globodera tabacum and other Globodera species, Heterodera avenae, Heterodera glycines, Heterodera schachtii, Heterodera trifolii, and other Heterodera species; seed gall nematodes, Anguina funesta, Anguina tritici and other Anguina species; stem and foliar nematodes, Aphelenchoides besseyi, Aphelenchoides fragariae, Aphelenchoides ritzemabosi and other Aphelenchoides species; sting nematodes, Belonolaimus longicaudatus and other Belonolaimus species; pine nematodes, Bursaphelenchus xylophilus and other Bursaphelenchus species; ring nematodes, Criconema species, Criconemella species, Criconemoides species, and Mesocriconema species; stem and bulb nematodes, Ditylenchus destructor, Ditylenchus dipsaci, Ditylenchus myceliophagus and other Ditylenchus species; awl nematodes, Dolichodorus species; spiral nematodes, Helicotylenchus dihystera, Helicotylenchus multicinctus and other Helicotylenchus species, Rotylenchus robustus and other Rotylenchus species; sheath nematodes, Hemicycliophora species and Hemicriconemoides species; Hirshmanniella species; lance nematodes, Hoplolaimus columbus, Hoplolaimus galeatus and other Hoplolaimus species; false root-knot nematodes, Nacobbus aberrans and other Nacobbus species; needle nematodes, Longidorus elongates and other Longidorus species; pin nematodes, Paratylenchus species; lesion nematodes, Pratylenchus brachyurus, Pratylenchus coffeae, Pratylenchus curvitatus, Pratylenchus goodeyi, Pratylencus neglectus, Pratylenchus penetrans, Pratylenchus scribneri, Pratylenchus vulnus, Pratylenchus zeae and other Pratylenchus species; Radinaphelenchus cocophilus and other Radinaphelenchus species; burrowing nematodes, Radopholus similis and other Radopholus species; reniform nematodes, Rotylenchulus reniformis and other Rotylenchulus species; Scutellonema species; stubby root nematodes, Trichodorus primitivus and other Trichodorus species; Paratrichodorus minor and other Paratrichodorus species; stunt nematodes, Tylenchorhynchus claytoni, Tylenchorhynchus dubius and other Tylenchorhynchus species and Merlinius species; citrus nematodes, Tylenchulus semipenetrans and other Tylenchulus species; dagger nematodes, Xiphinema americanum, Xiphinema index, Xiphinema diversicaudatum and other Xiphinema species; and other plant parasitic nematode species.

Moreover, the compounds of formula I and compositions containing them are especially useful for the control of insects and acarids of the orders Lepidoptera, Coleoptera, Diptera, Hemiptera, and Hymenoptera.

For use according to the present invention, the compounds of formula I can be converted into the customary formulations, e.g. solutions, emulsions, suspensions, dusts, powders, pastes, granules and directly sprayable solutions. The use form depends on the particular purpose and application method. Formulations and application methods are chosen to ensure in each case a fine and uniform distribution of the compound of the formula I according to the present invention.

The formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7514-0443-8), for example by extending the active compound with auxiliaries suitable for the formulation of agrochemicals, such as solvents and/or carriers, if desired emulsifiers, surfactants and dispersants, preservatives, antifoaming agents, anti-freezing agents, for seed treatment formulation also optionally colorants and/or binders and/or gelling agents.

Solvents/carriers, which are suitable, are e.g.:
solvents such as water, aromatic solvents (for example Solvesso products, xylene and the like), paraffins (for example mineral fractions), alcohols (for example methanol, butanol, pentanol, benzyl alcohol), ketones (for example cyclohexanone, gamma-butyrolactone), pyrrolidones (N-methyl-pyrrolidone (NMP), N-octylpyrrolidone NOP), acetates (glycol diacetate), alkyl lactates, lactones such as g-butyrolactone, glycols, fatty acid dimethylamides, fatty acids and fatty acid esters, triglycerides, oils of vegetable or animal origin and modified oils such as alkylated plant oils. In principle, solvent mixtures may also be used.
carriers such as ground natural minerals and ground synthetic minerals, such as silica gels, finely divided silicic acid, silicates, talc, kaolin, attaclay, limestone, lime, chalk, bole, loess, clay, dolomite, diatomaceous earth, calcium sulfate and magnesium sulfate, magnesium oxide, ground synthetic materials, fertilizers, such as, for example, ammonium sulfate, ammonium phosphate, ammonium nitrate, ureas and products of vegetable origin, such as cereal meal, tree bark meal, wood meal and nutshell meal, cellulose powders and other solid carriers.

Suitable emulsifiers are nonionic and anionic emulsifiers (for example polyoxyethylene fatty alcohol ethers, alkylsulfonates and arylsulfonates).

Examples of dispersants are lignin-sulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters.

Also anti-freezing agents such as glycerin, ethylene glycol, propylene glycol and bactericides such as can be added to the formulation.

Suitable antifoaming agents are for example antifoaming agents based on silicon or magnesium stearate.

Suitable preservatives are for example dichlorophen und benzyl alcohol hemiformal

Suitable thickeners are compounds, which confer a pseudoplastic flow behavior to the formulation, i.e. high viscosity at rest and low viscosity in the agitated stage. Mention may be made, in this context, for example, of commercial thickeners based on polysaccharides, such as Xanthan Gum® (Keizan® from Kelco), Rhodopol® 23 (Rhone Poulenc) or Veegum® (from R. T. Vanderbilt), or organic phyllosilicates, such as Attaclay® (from Engelhardt). Antifoam agents suitable for the dispersions according to the invention are, for example, silicone emulsions (such as, for example, Silikon® SRE, Wacker or Rhodorsil® from Rhodia), long-chain alcohols, fatty acids, organofluorine compounds and mixtures thereof. Biocides can be added to stabilize the compositions according to the invention against attack by microorganisms. Suitable biocides are, for example, based on isothiazolones such as the compounds marketed under the trademarks Proxel® from Avecia (or Arch) or Acticide® RS from Thor Chemie and Kathon® MK from Rohm & Haas. Suitable antifreeze agents are organic polyols, for example ethylene glycol, propylene glycol or glycerol. These are usually employed in amounts of not more than 10% by weight, based on the total weight of the active compound composition. If appropriate, the active compound compositions according to the invention may comprise 1 to 5% by weight of buffer, based on the total amount of the formulation prepared, to regulate the pH, the amount and type of the buffer used depending on the chemical properties of the active compound or the active compounds. Examples of buffers are alkali metal salts of weak inorganic or organic acids, such as, for example, phosphoric acid, boronic acid, acetic acid, propionic acid, citric acid, fumaric acid, tartaric acid, oxalic acid and succinic acid.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dusts can be prepared by mixing or concomitantly grinding the active substances with a solid carrier.

Granules, for example coated granules, impregnated granules and homogeneous granules, can be prepared by binding the active ingredients to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active ingredient. The active ingredients are employed in a purity of from 90% to 100%, preferably 95% to 100% (according to NMR spectrum).

For seed treatment purposes, respective formulations can be diluted 2-10 fold leading to concentrations in the ready to use preparations of 0.01 to 60% by weight active compound by weight, preferably 0.1 to 40% by weight.

The compound of formula I can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compounds according to the invention.

The following are examples of formulations:

1. Products for dilution with water for foliar application. For seed treatment purposes, such products may be applied to the seed diluted or undiluted.

A) Water-soluble concentrates (SL, LS)

10 parts by weight of the active compound is dissolved in 90 parts by weight of water or a water-soluble solvent. As an alternative, wetters or other auxiliaries are added. The active compound dissolves upon dilution with water, whereby a formulation with 10% (w/w) of active compound is obtained.

B) Dispersible concentrates (DC)

20 parts by weight of the active compound is dissolved in 70 parts by weight of cyclohexanone with addition of 10 parts by weight of a dispersant, for example polyvinylpyrrolidone. Dilution with water gives a dispersion, whereby a formulation with 20% (w/w) of active compounds is obtained.

C) Emulsifiable concentrates (EC)

15 parts by weight of the active compounds is dissolved in 7 parts by weight of xylene with addition of calcium dodecylbenzenesulfonate and castor oil ethoxylate (in each case 5 parts by weight). Dilution with water gives an emulsion, whereby a formulation with 15% (w/w) of active compounds is obtained.

D) Emulsions (EW, EO, ES)

25 parts by weight of the active compound is dissolved in 35 parts by weight of xylene with addition of calcium dodecylbenzenesulfonate and castor oil ethoxylate (in each case 5 parts by weight). This mixture is introduced into 30 parts by weight of water by means of an emulsifier machine (e.g. Ultraturrax) and made into a homogeneous emulsion. Dilution with water gives an emulsion, whereby a formulation with 25% (w/w) of active compound is obtained.

E) Suspensions (SC, OD, FS)

In an agitated ball mill, 20 parts by weight of the active compound is comminuted with addition of 10 parts by weight of dispersants, wetters and 70 parts by weight of water or of an organic solvent to give a fine active compound suspension. Dilution with water gives a stable suspension of the active compound, whereby a formulation with 20% (w/w) of active compound is obtained.

F) Water-dispersible granules and water-soluble granules (WG, SG)

50 parts by weight of the active compound is ground finely with addition of 50 parts by weight of dispersants and wetters and made as water-dispersible or water-soluble granules by means of technical appliances (for example extrusion, spray tower, fluidized bed). Dilution with water gives a stable dispersion or solution of the active compound, whereby a formulation with 50% (w/w) of active compound is obtained.

G) Water-dispersible powders and water-soluble powders (WP, SP, SS, WS)

75 parts by weight of the active compound are ground in a rotor-stator mill with addition of 25 parts by weight of dispersants, wetters and silica gel. Dilution with water gives a stable dispersion or solution of the active compound, whereby a formulation with 75% (w/w) of active compound is obtained.

H) Gel-Formulation (GF)

In an agitated ball mill, 20 parts by weight of the active compound is comminuted with addition of 10 parts by weight of dispersants, 1 part by weight of a gelling agent wetters and 70 parts by weight of water or of an organic solvent to give a fine active compound suspension. Dilution with water gives a stable suspension of the active compound, whereby a formulation with 20% (w/w) of active compound is obtained.

2. Products to be applied undiluted for foliar applications. For seed treatment purposes, such products may be applied to the seed diluted or undiluted.

I) Dustable powders (DP, DS)

5 parts by weight of the active compound are ground finely and mixed intimately with 95 parts by weight of finely divided kaolin. This gives a dustable product having 5% (w/w) of active compound.

J) Granules (GR, FG, GG, MG)

0.5 parts by weight of the active compound is ground finely and associated with 95.5 parts by weight of carriers, whereby a formulation with 0.5% (w/w) of active compound is obtained. Current methods are extrusion, spray-drying or the fluidized bed. This gives granules to be applied undiluted for foliar use.

K) ULV solutions (UL)

10 parts by weight of the active compound is dissolved in 90 parts by weight of an organic solvent, for example xylene. This gives a product having 10% (w/w) of active compound, which is applied undiluted for foliar use.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active ingredient concentrations in the ready-to-use products can be varied within relatively wide ranges. In general, they are from 0.0001 to 10% per weight, preferably from 0.01 to 1% per weight.

The active ingredients may also be used successfully in the ultra-low-volume process (ULV), it being possible to apply formulations comprising over 95% by weight of active ingredient, or even to apply the active ingredient without additives.

Activity of compounds against agricultural pests does not suggest their suitability for control of endo- and ectoparasites in and on animals which requires, for example, low, non-emetic dosages in the case of oral application, metabolic compatibility with the animal, low toxicity, and a safe handling.

Surprisingly it has now been found that compounds of formula I are suitable for combating endo- and ectoparasites in and on animals.

Compounds of formula I or the enantiomers or veterinarily acceptable salts thereof and compositions comprising them are preferably used for controlling and preventing infestations and infections animals including warm-blooded animals (including humans) and fish. They are for example suitable for controlling and preventing infestations and infections in mammals such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels.

Compounds of formula I or the enantiomers or veterinarily acceptable salts thereof and compositions comprising them are preferably used for controlling and preventing infestations and infections in domestic animals, such as dogs or cats.

Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of formula I or the enantiomers or veterinarily acceptable salts thereof and compositions comprising them are suitable for systemic and/or non-systemic control of ecto- and/or endoparasites. They are active against all or some stages of development.

The compounds of formula I are especially useful for combating ectoparasites.

The compounds of formula I are especially useful for combating parasites of the following orders and species, respectively:

fleas (Siphonaptera), e.g. Ctenocephalides felis, Ctenocephalides canis, Xenopsylla cheopis, Pulex irritans, Tunga penetrans, and Nosopsyllus fasciatus,

cockroaches (Blattaria—Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,

flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Chrysomya bezziana, Chrysomya hominivorax, Chrysomya macellaria, Chrysops discalis, Chrysops silacea, Chrysops atlanticus, Cochliomyia hominivorax, Cordylobia anthropophaga, Culicoides furens, Culex pipiens, Culex nigripalpus, Culex quinquefasciatus, Culex tarsalis, Culiseta inornata, Culiseta melanura, Dermatobia hominis, Fannia canicularis, Gasterophilus intestinalis, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucilia caprina, Lucilia cuprina, Lucilia sericata, Lycoria pectoralis, Mansonia spp., Musca domestica, Muscina stabulans, Oestrus ovis, Phlebotomus argentipes, Psorophora columbiae, Psorophora discolor, Prosimulium mixtum, Sarcophaga haemorrhoidalis, Sarcophaga sp., Simulium vittatum, Stomoxys calcitrans, Tabanus bovinus, Tabanus atratus, Tabanus lineola, and Tabanus similis,

lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.

ticks and parasitic mites (Parasitiformes): ticks (Ixodida), e.g. Ixodes scapularis, Ixodes holocyclus, Ixodes pacificus, Rhiphicephalus sanguineus, Dermacentor andersoni, Dermacentor variabilis, Amblyomma americanum, Ambryomma maculatum, Ornithodorus hermsi, Ornithodorus turicata and parasitic mites (Mesostigmata), e.g. Ornithonyssus bacoti and Dermanyssus gallinae,

Actinedida (Prostigmata) und Acaridida (Astigmata) e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp,

Bugs (Heteropterida): Cimex lectularius, Cimex hemipterus, Reduvius senilis, Triatoma spp., Rhodnius ssp., Panstrongylus ssp. and Arilus critatus,

Anoplurida, e.g. Haematopinus spp., Linognathus spp., Pediculus spp., Phtirus spp., and Solenopotes spp,

Mallophagida (suborders Arnblycerina and Ischnocerina), e.g. Trimenopon spp., Menopon spp., Trinoton spp., Bovicola spp., Werneckiella spp., Lepikentron spp., Trichodectes spp., and Felicola spp,

Roundworms Nematoda:

Wipeworms and Trichinosis (Trichosyringida), e.g. Trichinellidae (Trichinella spp.), (Trichuridae) Trichuris spp., Capillaria spp,

Rhabditida, e.g. Rhabditis spp, Strongyloides spp., Helicephalobus spp,

Strongylida, e.g. Strongylus spp., Ancylostoma spp., Necator americanus, Bunostomum spp. (Hookworm), Trichostrongylus spp., Haemonchus contortus., Ostertagia spp., Cooperia spp., Nematodirus spp., Dictyocaulus spp., Cyathostoma spp., Oesophagostomum spp., Stephanurus dentatus, Ollulanus spp., Chabertia spp., Stephanurus dentatus, Syngamus trachea, Ancylostoma spp., Uncinaria spp., Globocephalus spp., Necator spp., Metastrongylus spp., Muellerius capillaris, Protostrongylus spp., Angiostrongylus spp., Parelaphostrongylus spp. Aleurostrongylus abstrusus, and Dioctophyma renale,

Intestinal roundworms (Ascaridida), e.g. Ascaris lumbricoides, Ascaris suum, Ascaridia galli, Parascaris equorum, Enterobius vermicularis (Threadworm), Toxocara canis, Toxascaris leonine, Skrjabinema spp., and Oxyuris equi,

Camallanida, e.g. Dracunculus medinensis (guinea worm)

Spirurida, e.g. Thelazia spp. Wuchereria spp., Brugia spp., Onchocerca spp., Dirofilari spp.a, Dipetalonema spp., Setaria spp., Elaeophora spp., Spirocerca lupi, and Habronema spp.,

Thorny headed worms (Acanthocephala), e.g. Acanthocephalus spp., Macracanthorhynchus hirudinaceus and Oncicola spp,

Planarians (Plathelminthes):

Flukes (Trematoda), e.g. Faciola spp., Fascioloides magna, Paragonimus spp., Dicrocoelium spp., Fasciolopsis buski, Clonorchis sinensis, Schistosoma spp., Trichobilharzia spp., Alaria alata, Paragonimus spp., and Nanocyetes spp,

Cercomeromorpha, in particular Cestoda (Tapeworms), e.g. Diphyllobothrium spp., Tenia spp., Echinococcus spp., Dipylidium caninum, Multiceps spp., Hymenolepis spp., Mesocestoides spp., Vampirolepis spp., Moniezia spp., Anoplocephala spp., Sirometra spp., Anoplocephala spp., and Hymenolepis spp.

The compounds of formula I, and compositions containing them are particularly useful for the control of pests from the orders Diptera, Siphonaptera and Ixodida.

Moreover, the use of the compounds of formula I, and compositions containing them for combating mosquitoes is especially preferred.

The use of the compounds of formula I, and compositions containing them for combating flies is a further preferred embodiment of the present invention.

Furthermore, the use of the compounds of formula I, and compositions containing them for combating fleas is especially preferred.

The use of the compounds of formula I, and compositions containing them for combating ticks is a further preferred embodiment of the present invention.

The compounds of formula I also are especially useful for combating endoparasites (roundworms nematoda, thorny headed worms and planarians).

Administration can be carried out both prophylactically and therapeutically.

Administration of the active compounds is carried out directly or in the form of suitable preparations, orally, topically/dermally or parenterally.

For oral administration to warm-blooded animals, the formula I compounds may be formulated as animal feeds, animal feed premixes, animal feed concentrates, pills, solutions, pastes, suspensions, drenches, gels, tablets, boluses and capsules. In addition, the formula I compounds may be administered to the animals in their drinking water. For oral administration, the dosage form chosen should provide the animal with 0.01 mg/kg to 100 mg/kg of animal body weight per day of the formula I compound, preferably with 0.5 mg/kg to 100 mg/kg of animal body weight per day.

Alternatively, the formula I compounds may be administered to animals parenterally, for example, by intraruminal, intramuscular, intravenous or subcutaneous injection. The formula I compounds may be dispersed or dissolved in a physiologically acceptable carrier for subcutaneous injection. Alternatively, the formula I compounds may be formulated into an implant for subcutaneous administration. In addition the formula I compound may be transdermally administered to animals. For parenteral administration, the dosage form chosen should provide the animal with 0.01 mg/kg to 100 mg/kg of animal body weight per day of the formula I compound.

The formula I compounds may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the formula I compound. In addition, the formula I compounds may be formulated as ear tags for animals, particularly quadrupeds such as cattle and sheep.

Suitable preparations are:
Solutions such as oral solutions, concentrates for oral administration after dilution, solutions for use on the skin or in body cavities, pouring-on formulations, gels;
Emulsions and suspensions for oral or dermal administration; semi-solid preparations;
Formulations in which the active compound is processed in an ointment base or in an oil-in-water or water-in-oil emulsion base;
Solid preparations such as powders, premixes or concentrates, granules, pellets, tablets, boluses, capsules; aerosols and inhalants, and active compound-containing shaped articles.

Generally it is favorable to apply solid formulations which release compounds of formula I in total amounts of 10 mg/kg to 300 mg/kg, preferably 20 mg/kg to 200 mg/kg. The active compounds can also be used as a mixture with synergists or with other active compounds which act against pathogenic endo- and ectoparasites.

Compositions suitable for injection are prepared by dissolving the active ingredient in a suitable solvent and optionally adding further ingredients such as acids, bases, buffer salts, preservatives, and solubilizers. The solutions are filtered and filled sterile.

Suitable solvents are physiologically tolerable solvents such as water, alkanols such as ethanol, butanol, benzyl alcohol, glycerol, propylene glycol, polyethylene glycols, N-methyl-pyrrolidone, 2-pyrrolidone, and mixtures thereof.

The active compounds can optionally be dissolved in physiologically tolerable vegetable or synthetic oils, which are suitable for injection.

Suitable solubilizers are solvents, which promote the dissolution of the active compound in the main solvent or prevent its precipitation. Examples are polyvinylpyrrolidone, polyvinyl alcohol, polyoxyethylated castor oil, and polyoxyethylated sorbitan ester.

Suitable preservatives are benzyl alcohol, trichlorobutanol, p-hydroxybenzoic acid esters, and n-butanol.

Oral solutions are administered directly. Concentrates are administered orally after prior dilution to the use concentration. Oral solutions and concentrates are prepared according to the state of the art and as described above for injection solutions, sterile procedures not being necessary.

Solutions for use on the skin are trickled on, spread on, rubbed in, sprinkled on or sprayed on.

Solutions for use on the skin are prepared according to the state of the art and according to what is described above for injection solutions, sterile procedures not being necessary.

Further suitable solvents are polypropylene glycol, phenyl ethanol, phenoxy ethanol, ester such as ethyl or butyl acetate, benzyl benzoate, ethers such as alkyleneglycol alkylether, e.g. dipropylenglycol monomethylether, ketons such as acetone, methylethylketone, aromatic hydrocarbons, vegetable and synthetic oils, dimethylformamide, dimethylacetamide, transcutol, solketal, propylencarbonate, and mixtures thereof.

It may be advantageous to add thickeners during preparation. Suitable thickeners are inorganic thickeners such as bentonites, colloidal silicic acid, aluminium monostearate, organic thickeners such as cellulose derivatives, polyvinyl alcohols and their copolymers, acrylates and methacrylates.

Gels are applied to or spread on the skin or introduced into body cavities. Gels are prepared by treating solutions, which have been prepared as described in the case of the injection solutions with sufficient thickener that a clear material having an ointment-like consistency results. The thickeners employed are the thickeners given above.

Pour-on formulations are poured or sprayed onto limited areas of the skin, the active compound penetrating the skin and acting systemically.

Pour-on formulations are prepared by dissolving, suspending or emulsifying the active compound in suitable skin-compatible solvents or solvent mixtures. If appropriate, other auxiliaries such as colorants, bioabsorption-promoting substances, antioxidants, light stabilizers, adhesives are added.

Suitable solvents which are: water, alkanols, glycols, polyethylene glycols, polypropylene glycols, glycerol, aromatic alcohols such as benzyl alcohol, phenylethanol, phenoxyethanol, esters such as ethyl acetate, butyl acetate, benzyl benzoate, ethers such as alkylene glycol alkyl ethers such as dipropylene glycol monomethyl ether, diethylene glycol mono-butyl ether, ketones such as acetone, methyl ethyl ketone, cyclic carbonates such as propylene carbonate, ethylene carbonate, aromatic and/or aliphatic hydrocarbons, vegetable or synthetic oils, DMF, dimethylacetamide, n-alkylpyrrolidones such as methylpyrrolidone, n-butylpyrrolidone or n-octylpyrrolidone, N-methylpyrrolidone, 2-pyrrolidone, 2,2-dimethyl-4-oxy-methylene-1,3-dioxolane and glycerol formal.

Suitable colorants are all colorants permitted for use on animals and which can be dissolved or suspended.

Suitable absorption-promoting substances are, for example, DMSO, spreading oils such as isopropyl myristate, dipropylene glycol pelargonate, silicone oils and copolymers thereof with polyethers, fatty acid esters, triglycerides, fatty alcohols.

Suitable antioxidants are sulfites or metabisulfites such as potassium metabisulfite, ascorbic acid, butylhydroxytoluene, butylhydroxyanisole, tocopherol.

Suitable light stabilizers are, for example, novantisolic acid.

Suitable adhesives are, for example, cellulose derivatives, starch derivatives, polyacrylates, natural polymers such as alginates, gelatin.

Emulsions can be administered orally, dermally or as injections.

Emulsions are either of the water-in-oil type or of the oil-in-water type.

They are prepared by dissolving the active compound either in the hydrophobic or in the hydrophilic phase and homogenizing this with the solvent of the other phase with the aid of suitable emulsifiers and, if appropriate, other auxiliaries such as colorants, absorption-promoting substances, preservatives, antioxidants, light stabilizers, viscosity-enhancing substances.

Suitable hydrophobic phases (oils) are:

liquid paraffins, silicone oils, natural vegetable oils such as sesame oil, almond oil, castor oil, synthetic triglycerides such as caprylic/capric biglyceride, triglyceride mixture with vegetable fatty acids of the chain length C₈-C₁₂ or other specially selected natural fatty acids, partial glyceride mixtures of saturated or unsaturated fatty acids possibly also containing hydroxyl groups, mono- and diglycerides of the C₈-C₁₀ fatty acids, fatty acid esters such as ethyl stearate, di-n-butyryl adipate, hexyl laurate, dipropylene glycol perlargonate, esters of a branched fatty acid of medium chain length with saturated fatty alcohols of chain length C₁₆-C₁₈, isopropyl myristate, isopropyl palmitate, caprylic/capric acid esters of saturated fatty alcohols of chain length C₁₂-C₁₈, isopropyl stearate, oleyl oleate, decyl oleate, ethyl oleate, ethyl lactate, waxy fatty acid esters such as synthetic duck coccygeal gland fat, dibutyl phthalate, diisopropyl adipate, and ester mixtures related to the latter, fatty alcohols such as isotridecyl alcohol, 2-octyidodecanol, cetylstearyl alcohol, oleyl alcohol, and fatty acids such as oleic acid and mixtures thereof.

Suitable hydrophilic phases are: water, alcohols such as propylene glycol, glycerol, sorbitol and mixtures thereof.

Suitable emulsifiers are:

non-ionic surfactants, e.g. polyethoxylated castor oil, polyethoxylated sorbitan monooleate, sorbitan monostearate, glycerol monostearate, polyoxyethyl stearate, alkylphenol polyglycol ether;

ampholytic surfactants such as di-sodium N-lauryl-p-iminodipropionate or lecithin; anionic surfactants, such as sodium lauryl sulfate, fatty alcohol ether sulfates, mono/dialkyl polyglycol ether orthophosphoric acid ester monoethanolamine salt; cation-active surfactants, such as cetyltrimethylammonium chloride.

Suitable further auxiliaries are: substances which enhance the viscosity and stabilize the emulsion, such as carboxymethylcellulose, methylcellulose and other cellulose and starch derivatives, polyacrylates, alginates, gelatin, gum arabic, polyvinylpyrrolidone, polyvinyl alcohol, copolymers of methyl vinyl ether and maleic anhydride, polyethylene glycols, waxes, colloidal silicic acid or mixtures of the substances mentioned.

Suspensions can be administered orally or topically/dermally. They are prepared by suspending the active compound in a suspending agent, if appropriate with addition of other auxiliaries such as wetting agents, colorants, bioabsorption-promoting substances, preservatives, antioxidants, light stabilizers.

Liquid suspending agents are all homogeneous solvents and solvent mixtures.

Suitable wetting agents (dispersants) are the emulsifiers given above.

Other auxiliaries, which may be mentioned are those given above.

Semi-solid preparations can be administered orally or topically/dermally. They differ from the suspensions and emulsions described above only by their higher viscosity.

For the production of solid preparations, the active compound is mixed with suitable excipients, if appropriate with addition of auxiliaries, and brought into the desired form.

Suitable excipients are all physiologically tolerable solid inert substances. Those used are inorganic and organic substances. Inorganic substances are, for example, sodium chloride, carbonates such as calcium carbonate, hydrogencarbonates, aluminium oxides, titanium oxide, silicic acids, argillaceous earths, precipitated or colloidal silica, or phosphates. Organic substances are, for example, sugar, cellulose, foodstuffs and feeds such as milk powder, animal meal, grain meals and shreds, starches.

Suitable auxiliaries are preservatives, antioxidants, and/or colorants, which have been mentioned above.

Other suitable auxiliaries are lubricants and glidants such as magnesium stearate, stearic acid, talc, bentonites, disintegration-promoting substances such as starch or crosslinked polyvinylpyrrolidone, binders such as starch, gelatin or linear polyvinylpyrrolidone, and dry binders such as microcrystalline cellulose.

In general, “parasiticidally effective amount” means the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions used in the invention. A parasiticidally effective amount of the compositions will also vary according to the prevailing conditions such as desired parasiticidal effect and duration, target species, mode of application, and the like.

The compositions, which can be used in the invention can comprise generally from about 0.001 to 95% of the compound of formula I.

Generally it is favorable to apply the compounds of formula I in total amounts of 0.5 mg/kg to 100 mg/kg per day, preferably 1 mg/kg to 50 mg/kg per day.

Ready-to-use preparations contain the compounds acting against parasites, preferably ectoparasites, in concentrations of 10 ppm to 80 percent by weight, preferably from 0.1 to 65 percent by weight, more preferably from 1 to 50 percent by weight, most preferably from 5 to 40 percent by weight.

Preparations, which are diluted before use, contain the compounds acting against ectoparasites in concentrations of 0.5 to 90 percent by weight, preferably of 1 to 50 percent by weight.

Furthermore, the preparations comprise the compounds of formula I against endoparasites in concentrations of 10 ppm to 2 percent by weight, preferably of 0.05 to 0.9 percent by weight, very particularly preferably of 0.005 to 0.25 percent by weight.

In a preferred embodiment of the present invention, the compositions comprising the compounds of formula I them are applied dermally/topically.

In a further preferred embodiment, the topical application is conducted in the form of compound-containing shaped articles such as collars, medallions, ear tags, bands for fixing at body parts, and adhesive strips and foils.

Generally it is favorable to apply solid formulations which release compounds of formula I in total amounts of 10 mg/kg to 300 mg/kg, preferably 20 mg/kg to 200 mg/kg, most preferably 25 mg/kg to 160 mg/kg body weight of the treated animal in the course of three weeks.

For the preparation of the shaped articles, thermoplastic and flexible plastics as well as elastomers and thermoplastic elastomers are used. Suitable plastics and elastomers are polyvinyl resins, polyurethane, polyacrylate, epoxy resins, cellulose, cellulose derivatives, polyamides and polyester which are sufficiently compatible with the compounds of formula I. A detailed list of plastics and elastomers as well as preparation procedures for the shaped articles is given e.g. in WO 03/086075.

Compositions of this invention may also contain other active ingredients, for example other pesticides, insecticides, herbicides, fertilizers such as ammonium nitrate, urea, potash, and superphosphate, phytotoxicants and plant growth regulators, safeners and nematicides. These additional ingredients may be used sequentially or in combination with the above-described compositions, if appropriate also added only immediately prior to use (tank mix). These agents usually are admiexed with the agents according to the invention in a weight ratio of 1:10 to 10:1. For example, the plant(s) may be sprayed with a composition of this invention either before or after being treated with other active ingredients.

The following list M of pesticides together with which the compounds according to the invention can be used, is intended to illustrate the possible combinations, but not to impose any limitation:

M.1. Organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;

M.2. Carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;

M.3. Pyrethroids: allethrin, bifenthrin, cyfluthrin, cyhalothrin, cyphenothrin, cypermethrin, alpha-cypermethrin, beta-cypermethrin, zeta-cypermethrin, deltamethrin, empenthrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin;

M.4. Growth regulators: a) chitin synthesis inhibitors: benzoylureas: chlorfluazuron, diflubenzuron, flucycloxuron, flufenoxuron, hexaflumuron, lufenuron, novaluron, teflubenzuron, triflumuron; buprofezin, diofenolan, hexythiazox, etoxazole, clofentazine; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramat;

M.5. Nicotinic receptor agonists/antagonists compounds: clothianidin, dinotefuran, imidacloprid, thiamethoxam, nitenpyram, acetamiprid, thiacloprid; the thiazol compound of formula (Γ¹)

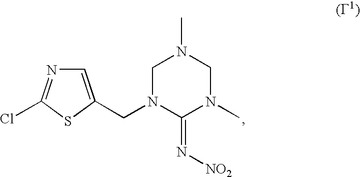

M.6. GABA antagonist compounds: acetoprole, endosulfan, ethiprole, fipronil, vaniliprole, pyrafluprole, pyriprole, the phenylpyrazole compound of formula Γ²

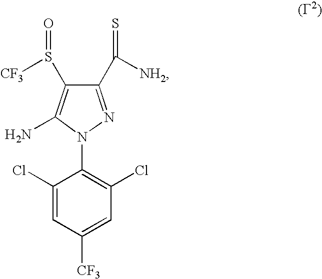

M.7. Macrocyclic lactone insecticides: abamectin, emamectin, milbemectin, lepimectin, spinosad;

M.8. METI I compounds: fenazaquin, pyridaben, tebufenpyrad, tolfenpyrad, flufenerim;

M.9. METI II and III compounds: acequinocyl, fluacyprim, hydramethylnon;

M.10. Uncoupler compounds: chlorfenapyr;

M.11. Oxidative phosphorylation inhibitor compounds: cyhexatin, diafenthiuron, fenbutatin oxide, propargite;

M.12. Moulting disruptor compounds: cyromazine;

M.13. Mixed Function Oxidase inhibitor compounds: piperonyl butoxide;

M.14. Sodium channel blocker compounds: indoxacarb, metaflumizone,

M.15. Various: amitraz, benclothiaz, bifenazate, cartap, flonicamid, pyridalyl, pymetrozine, sulfur, thiocyclam, flubendiamide, cyenopyrafen, flupyrazofos, cyflumetofen, amidoflumet, the aminoquinazolinone compound of formula Γ⁴

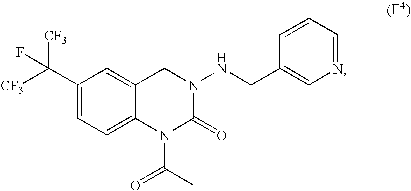

N—R′-2,2-dihalo-1-R″cyclo-propanecarboxamide-2-(2,6-dichloro-α,α,α-tri-fluoro-p-tolyl)hydrazone or N—R′-2,2-di(R′″)propionamide-2-(2,6-dichloro-α,α,α-trifluoro-p-tolyl)-hydrazone, wherein R′ is methyl or ethyl, halo is chloro or bromo, R″ is hydrogen or methyl and R′″ is methyl or ethyl, anthranilamide compounds of formula Γ⁵

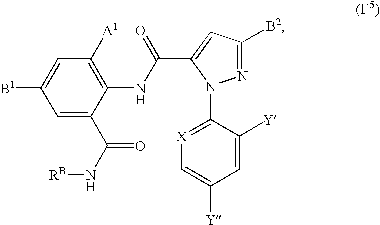

wherein A¹ is CH₃, Cl, Br, I, X is C—H, C—Cl, C—F or N, Y′ is F, Cl, or Br, Y″ is H, F, Cl, CF₃, B¹ is hydrogen, Cl, Br, I, CN, B² is Cl, Br, CF₃, OCH₂CF₃, OCF₂H, or OCF₂CHFOCF₃ and R^{B} is hydrogen, CH₃ or CH(CH₃)₂, the aminoisothiazole compound of formula (Γ⁶)

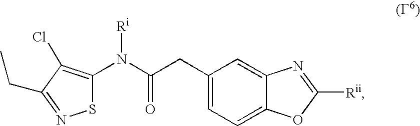

wherein Rⁱ is —CH₂OCH₂CH₃ or H and Rⁱⁱ is CF₂CF₂CF₃ or CH₂CH(CH₃)₃, and malononitrile compounds as described in JP 2002 284608, WO 02/89579, WO 02/90320, WO 02/90321, WO 04/06677, WO 04/20399, JP 2004 99597, WO 05/68423, WO 05/68432, or WO 05/63694, especially the malononitrile compounds CF₃(CH₂)₂C(CN)₂CH₂(CF₂)₃CF₂H, CF₃(CH₂)₂C(CN)₂CH₂(CF₂)₅CF₂H, CF₃(CH₂)₂C(CN)₂(CH₂)₂C(CF₃)₂F, CF₃(CH₂)₂C(CN)₂(CH₂)₂(CF₂)₃CF₃, CF₂H(CF₂)₃CH₂C(CN)₂CH₂(CF₂)₃CF₂H, CF₃(CH₂)₂C(CN)₂CH₂(CF₂)₃CF₃, CF₃(CF₂)₂CH₂C(CN)₂CH₂(CF₂)₃CF₂H, and CF₃CF₂CH₂C(CN)₂CH₂(CF₂)₃CF₂H.

The commercially available compounds of the group A may be found in The Pesticide Manual, 13^{th} Edition, British Crop Protection Council (2003) among other publications. Thioamides of formula Γ² and their preparation have been described in WO 98/28279. Lepimectin is known from Agro Project, PJB Publications Ltd, November 2004. Benclothiaz and its preparation have been described in EP-A1 454621. Methidathion and Paraoxon and their preparation have been described in Farm Chemicals Handbook, Volume 88, Meister Publishing Company, 2001. Acetoprole and its preparation have been described in WO 98/28277. Metaflumizone and its preparation have been described in EP-A1 462 456. Flupyrazofos has been described in Pesticide Science 54, 1988, p. 237-243 and in U.S. Pat. No. 4,822,779. Pyrafluprole and its preparation have been described in JP 2002193709 and in WO 01/00614. Pyriprole and its preparation have been described in WO 98/45274 and in U.S. Pat. No. 6,335,357. Amidoflumet and its preparation have been described in U.S. Pat. No. 6,221,890 and in JP 21010907. Flufenerim and its preparation have been described in WO 03/007717 and in WO 03/007718. Cyflumetofen and its preparation have been described in WO 04/080180. The aminoquinazolinone compound of formula Γ⁴ has been described in EP A 109 7932 Anthranilamides of formula Γ⁵ and their preparation have been described in WO 01/70671; WO 02/48137; WO 03/24222, WO 03/15518, WO 04/67528; WO 04/33468; and WO 05/118552. The malononitrile compounds CF₃(CH₂)₂C(CN)₂CH₂(CF₂)₃CF₂H, CF₃(CH₂)₂C(CN)₂CH₂(CF₂)₅CF₂H, CF₃(CH₂)₂C(CN)₂(CH₂)₂C(CF₃)₂F, CF₃(CH₂)₂C(CN)₂(CH₂)₂(CF₂)₃CF₃, CF₂H(CF₂)₃CH₂C(CN)₂CH₂(CF₂)₃CF₂H, CF₃(CH₂)₂C(CN)₂CH₂(CF₂)₃CF₃, CF₃(CF₂)₂CH₂C(CN)₂CH₂(CF₂)₃CF₂H, and CF₃CF₂CH₂C(CN)₂CH₂(CF₂)₃CF₂H have been described in WO 05/63694.

In the methods according to the invention the pests are controlled by contacting the target parasite/pest, its food supply, habitat, breeding ground or its locus with a pesticidally effective amount of compounds of formula I or with a salt thereof or with a composition, containing a pesticidally effective amount of a compound of formula I or a salt thereof.

“Locus” means a habitat, breeding ground, plant, seed, soil, area, material or environment in which a pest or parasite is growing or may grow.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

The compounds of the invention can also be applied preventively to places at which occurrence of the pests is expected.

The compounds of formula I may be also used to protect growing plants from attack or infestation by pests by contacting the plant with a pesticidally effective amount of compounds of formula I. As such, “contacting” includes both direct contact (applying the compounds/compositions directly on the pest and/or plant—typically to the foliage, stem or roots of the plant) and indirect contact (applying the compounds/compositions to the locus of the pest and/or plant).

The aforementioned compositions are particularly useful for protecting crop plants against infestation of said pests or for combating these pests in infested plants.

For use in treating crop plants, the rate of application of the active ingredients of this invention may be in the range of 0.1 g to 4000 g per hectare, desirably from 25 g to 600 g per hectare, more desirably from 50 g to 500 g per hectare.

In accordance with one variant of the present invention, a further subject of the invention is a method of treating soil by the application, in particular into the seed drill, e.g. in form of a granular formulation containing compounds of formula I alone or in mixture with other active ingredients. This method is advantageously employed in seedbeds of cereal, maize, cotton and sunflower. For cereals and maize, the rates of application may depend from the active ingredients used, and may range between 50 and 500 per hectare for one active ingredient and between 50 and 200 g per hectare for the other active ingredient.

In the case of soil treatment or of application to the pests dwelling place or nest, the quantity of active ingredient ranges from 0.0001 to 500 g per 100 m², preferably from 0.001 to 20 g per 100 m².

The compounds of formula I are also suitable for the treatment of seeds in order to protect the seed from insect pest, in particular from soil-living insect pests and the resulting plant's roots and shoots against soil pests and foliar insects.

The compounds of formula I are particularly useful for the protection of the seed from soil pests and the resulting plant's roots and shoots against soil pests and foliar insects. The protection of the resulting plant's roots and shoots is preferred. More preferred is the protection of resulting plant's shoots from piercing and sucking insects, wherein the protection from aphids is most preferred.

The present invention therefore comprises a method for the protection of seeds from insects, in particular from soil insects and of the seedlings' roots and shoots from insects, in particular from soil and foliar insects, said method comprising contacting the seeds before sowing and/or after pregermination with a compound of the general formula I or a salt thereof. Particularly preferred is a method, wherein the plant's roots and shoots are protected, more preferably a method, wherein the plants shoots are protected form piercing and sucking insects, most preferably aa method, wherein the plants shoots are protected from aphids.

The term seed embraces seeds and plant propagules of all kinds including but not limited to true seeds, seed pieces, suckers, corms, bulbs, fruit, tubers, grains, cuttings, cut shoots and the like and means in a preferred embodiment true seeds.

The term seed treatment comprises all suitable seed treatment techniques known in the art, such as seed dressing, seed coating, seed dusting, seed soaking and seed pelleting.

The present invention also comprises seeds coated with or containing the active compound.

The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

Suitable seed is seed of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In addition, the active compound may also be used for the treatment seeds from plants, which tolerate the action of herbicides or fungicides or insecticides owing to breeding, including genetic engineering methods.

For example, the active compound can be employed in treatment of seeds from plants, which are resistant to herbicides from the group consisting of the sulfonylureas, imidazolinones, glufosinate-ammonium or glyphosate-isopropylammonium and analogous active substances (see for example, EP-A-0242236, EP-A-242246) (WO 92/00377) (EP-A-0257993, U.S. Pat. No. 5,013,659) or in transgenic crop plants, for example cotton, with the capability of producing Bacillus thuringiensis toxins (Bt toxins) which make the plants resistant to certain pests (EP-A-0142924, EP-A-0193259),

Furthermore, the active compound can be used also for the treatment of seeds from plants, which have modified characteristics in comparison with existing plants consist, which can be generated for example by traditional breeding methods and/or the generation of mutants, or by recombinant procedures). For example, a number of cases have been described of recombinant modifications of crop plants for the purpose of modifying the starch synthesized in the plants (e.g. WO 92/11376, WO 92/14827, WO 91/19806) or of transgenic crop plants having a modified fatty acid composition (WO 91/13972).

The seed treatment application of the active compound is carried out by spraying or by dusting the seeds before sowing of the plants and before emergence of the plants.

In the treatment of seeds the corresponding formulations are applied by treating the seeds with an effective amount of the active compound. Herein, the application rates of the active compound are generally from 0.1 g to 10 kg per 100 kg of seed, preferably from 1 g to 5 kg per 100 kg of seed, in particular from 1 g to 2.5 kg per 100 kg of seed. For specific crops such as lettuce the rate can be higher.

Compositions, which are especially useful for seed treatment are e.g.:

A Soluble concentrates (SL, LS)

D Emulsions (EW, EO, ES)

E Suspensions (SC, OD, FS)

F Water-dispersible granules and water-soluble granules (WG, SG)

G Water-dispersible powders and water-soluble powders (WP, SP, WS)

H Gel-Formulations (GF)

I Dustable powders (DP, DS)

Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter

In a preferred embodiment a FS formulation is used for seed treatment. Typically, a FS formulation may comprise 1-800 g/l of active ingredient, 1-200 g/l Surfactant, 0 to 200 g/l antifreezing agent, 0 to 400 g/l of binder, 0 to 200 g/l of a pigment and up to 1 liter of a solvent, preferably water.

Preferred FS formulations of compounds of formula I for seed treatment usually comprise from 0.1 to 80% by weight (1 to 800 g/L) of the active ingredient, from 0.1 to 20% by weight (1 to 200 g/L) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight.

Seed Treatment formulations may additionally also comprise binders and optionally colorants.

Binders can be added to improve the adhesion of the active materials on the seeds after treatment. Suitable binders are block copolymers EO/PO surfactants but also polyvinylalcoholsl, polyvinylpyrrolidones, polyacrylates, polymethacrylates, polybutenes, polyisobutylenes, polystyrene, polyethyleneamines, polyethyleneamides, polyethyleneimines (Lupasol®, Polymin®), polyethers, polyurethans, polyvinylacetate, tylose and copolymers derived from these polymers.

Optionally, also colorants can be included in the formulation. Suitable colorants or dyes for seed treatment formulations are Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of a gelling agent is carrageen (Satiagel®)

In the treatment of seed, the application rates of the compounds I are generally from 0.1 g to 10 kg per 100 kg of seed, preferably from 1 g to 5 kg per 100 kg of seed, in particular from 1 g to 1000 g per 100 kg of seed.

The invention therefore also relates to seed comprising a compound of the formula I, or an agriculturally useful salt of 1, as defined herein. The amount of the compound I or the agriculturally useful salt thereof will in general vary from 0.1 g to 10 kg per 100 kg of seed, preferably from 1 g to 5 kg per 100 kg of seed, in particular from 1 g to 1000 g per 100 kg of seed.

The compounds of formula I are gernerally effective through both contact and ingestion.

According to a preferred embodiment of the invention, the compounds of formula I are employed via soil application. Soil application is especially favorable for use against ants, termites, crickets, or cockroaches.

The compounds of the invention may therefor also be applied against non-crop insect pests, such as ants, termites, wasps, flies, mosquitos, crickets, or cockroaches. For use against said non-crop pests, compounds of formula I are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickyness, moisture retention or aging characteristics.

The bait employed in the composition is a product, which is sufficiently attractive to incite insects such as ants, termites, wasps, flies, mosquitos, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

For use in bait compositions, the typical content of active ingredient is from 0.001 weight % to 15 weight %, desirably from 0.001 weight % to 5% weight % of active compound. The composition used may also comprise other additives such as a solvent of the active material, a flavoring agent, a preserving agent, a dye or a bitter agent. Its attractiveness may also be enhanced by a special color, shape or texture.

Formulations of compounds of formula I as aerosols (e.g in spray cans), oil sprays or pump sprays are highly suitable for the non-professional user for controlling pests such as flies, fleas, ticks, mosquitos or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylformamide, N-methylpyrrolidone, dimethyl sulfoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

For use in spray compositions, the content of active ingredient is from 0.001 to 80 weights %, preferably from 0.01 to 50 weight % and most preferably from 0.01 to 15 weight %.

The compounds of formula I and its respective compositions can also be used in mosquito and fumigating coils, smoke cartridges, vaporizer plates or long-term vaporizers and also in moth papers, moth pads or other heat-independent vaporizer systems.

The present invention is now illustrated in further details by the following examples.

### SYNTHESIS EXAMPLES

With due modification of the starting compounds, the protocols shown in the synthesis examples below were used for obtaining further compounds I.

The obtained compounds I are listed in Table I.

Starting materials such as (3,3,3-trifluoropropyl)malononitrile, (3,4,4-trifluoro-3-butenyl)malononitrile or (3-butenyl)malononitrile can be obtained following procedures described in WO 04/006677, WO 02/090320 or WO 02/089579, respectively.

The products were characterized by coupled High Performance Liquid Chromatography/mass spectrometry (HPLC/MS), by NMR or by their melting points. Analytical HPLC column: RP-18 column Chromolith Speed ROD from Merck KgaA, Germany). Elution: acetonitrile+0.1% trifluoroacetic acid (TFA)/water+0.1% trifluoroacetic acid (TFA) in a ratio of from 5:95 to 95:5 in 5 minutes at 40° C.

MS: Quadrupol electrospray ionisation, 80 V (positiv modus)

The conditions for preparative HPLC were as follows: Purospher Star RP18e Hibar RT 75-25 column (3 μm), elution: acetonitrile+0.1% trifluoroacetic acid (TFA)/water+0.1% trifluoroacetic acid (TFA) in a ratio of from 20:80 to 100:0 in 13 minutes, detection by UV at 205 nm, 214 nm, 254 nm, 280 nm and 400 nm or by MS.

### Synthesis Example 1

### Preparation of 3,3-dicyano-6,6,6-trifluoro-hexanoic acid tert-butyl-methyl-amide (compound I-1)

A mixture of 164 mg (1 mmol) 2-chloro-N-methyl-N-tert-butyl acetamide and 276 mg (2 mmol) potassium carbonate in 6 ml DMF was stirred at 20-25° C. for 1 hour under an argon atmosphere. Subsequently, 162 mg (1 mmol) 3,3,3-trifluoropropyl)malononitrile were added and the mixture was stirred overnight at 20-25° C. After evaporation of volatile materials the remaining crude product was dissolved in CH₂Cl₂, washed with 15 ml water (2×) and dried over Mg₂SO₄. After evaporation of the solvent, 281 mg (1 mmol, 100%) of compound I-1 were obtained. (HPLC/MS: RT=3.190 min, m/z=290 [M+H]⁺).

### Synthesis Example 2

### Preparation of 3,3-dicyano-6,6,6-trifluoro-hexanoic acid (cyano-dimethyl-methyl)-methyl-amide (compound I-2)

According to the procedures described in example 1, 191 mg (0.6 mmol, 62%) of compound I-2 were obtained from 175 mg (1 mmol) 2-chloro-N-(cyano-dimethyl-methyl)-N-methyl acetamide and 1 mmol 3,3,3-trifluoropropyl)malononitrile. (HPLC/MS: RT=2.694 min, m/z=301 [M+H]⁺).

In the following table 1, compounds of formula (I)

TABLE I

| | |
|---|---|
| | (I) |
| 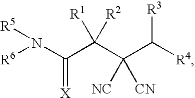 | |
| are characterized, wherein | |
| R⁵ and R⁶ are defined as in table I, | |
| X is O, | |
| R¹, R² and R³ are H and | |
| R⁴ is CF₃. | |

| | | | Physical data |
|---|---|---|---|
| | | | ¹H-NMR (d₆-DMSO, δ[ppm]); |
| | | | HPLC/MS |
| No. | R⁵ | R⁶ | (RT [min]; m/z [M + H]⁺; |
| I-1 | CH₃ | C(CH₃)₃ | RT = 3.190 min, m/z = 290 |
| | | | [M + H]⁺ |
| I-2 | CH₃ | C(CH₃)₂CN | RT = 2.694 min, m/z = 301 |
| | | | [M + H]⁺ |
| I-3 | CH₂CH₂OCH₃ | CH₂CH₂CN | RT = 2.685 min, m/z = 331 |
| | | | [M + H]⁺ |
| I-4 | CH₂CH₂CH₃ | CH₂CH₂CN | RT = 2.777 min, m/z = 315 |
| | | | [M + H]⁺ |
| I-5 | CH₂CH₂OCH₃ | CH₂CN | RT = 2.145 min, m/z = 317 |
| | | | [M + H]⁺ |
| I-6 | CH(CH₃)₂ | CH(CH₃)CN | RT = 3.133 min, m/z = 315 |
| | | | [M + H]⁺ |
| I-7 | CH₃ | CH₃ | RT = 2.130 min, m/z = 248 |
| | | | [M + H]⁺ |
| I-8 | C(CH₃)₃ | H | RT = 2.577 min, m/z = 276 |
| | | | [M + H]⁺ |
| I-9 | cyclo-C₆H₁₁ | CH₂CH₂CN | RT = 3.207 min, m/z = 355 |
| | | | [M + H]⁺ |
| I-10 | CH(CH₃)₂ | CH₂CH₂CN | RT = 2.720 min, m/z = 315 |
| | | | [M + H]⁺ |
| I-11 | (CH₂)₂O(CH₂)₂ | | RT = 2.191 min, m/z = 290 |
| | | | [M + H]⁺ |
| I-12 | CH₃ | 3,4-Cl₂—C₆H₃ | RT = 3.514 min, m/z = 378 |
| | | | [M + H]⁺ |

Examples for the action against harmful pests:

1. Activity against Boll weevil (Anthonomus grandis)

The active compounds were formulated as a 2500 ppm solution in 1:3 DMSO:water. 10 to 15 eggs were placed into microtiterplates filled with 2% agar-agar in water and 300 ppm formaline. The eggs were sprayed with 20 μl of the test solution, the plates were sealed with pierced foils and kept at 24-26° C. and 75-85% humidity with a day/night cycle for 3 to 5 days. Mortality was assessed on the basis of the remaining unhatched eggs or larvae on the agar surface and/or quantity and depth of the digging channels caused by the hatched larvae. Tests were replicated 2 times.

In this test, compound I-1 at 2500 ppm showed over 75% mortality compared to 0% mortality of untreated controls.

2. Activity against Mediterranean fruitfly (Ceratitis capitata)

The active compounds are formulated as a 2500 ppm solution in 1:3 DMSO:water. 50 to 80 eggs are placed into microtiterplates filled with 0.5% agar-agar and 14% diet in water. The eggs are sprayed with 5 μl of the test solution, the plates are sealed with pierced foils and kept at 27-29° C. and 75-85% humidity under fluorescent light for 6 days. Mortality is assessed on the basis of the agility of the hatched larvae. Tests are replicated 2 times.

3. Activity against Tobacco budworm (Heliothis virescens)

The active compounds are formulated as a 2500 ppm solution in 1:3 DMSO:water. 15 to 25 eggs are placed into microtiterplates filled with diet. The eggs are sprayed with 10 μl of the test solution, the plates are sealed with pierced foils and kept at 27-29° C. and 75-85% humidity under fluorescent light for 6 days. Mortality is assessed on the basis of the agility and of comparative feeding of the hatched larvae. Tests are replicated 2 times.

4. Activity against Vetch aphid (Megoura viciae)

The active compounds were formulated as a 2500 ppm solution in 1:3 DMSO:water. Bean leaf disks were placed into microtiterplates filled with 0.8% agar-agar and 2.5 ppm OPUS™. The leaf disks were sprayed with 2.5 μl of the test solution and 5 to 8 adult aphids were placed into the microtiterplates which were then closed and kept at 22-24° C. and 35-45% under fluorescent light for 6 days. Mortality was assessed on the basis of vital, reproduced aphids. Tests were replicated 2 times.

In this test, compounds I-1 and 1-6 at 2500 ppm showed over 75% mortality compared to 0% mortality of untreated controls.

5. Activity against Wheat aphid (Rhopalosiphum padi)

The active compounds are formulated as a 2500 ppm solution in 1:3 DMSO:water. Barlay leaf disk are placed into microtiterplates filled with 0.8% agar-agar and 2.5 ppm OPUS™.The leaf disks are sprayed with 2.5 μl of the test solution and 3 to 8 adult aphids are placed into the microtiterplates which are then closed and kept at 22-24° C. and 35-45% humidity under fluorescent light for 5 days. Mortality is assessed on the basis of vital aphids. Tests are replicated 2 times.

6. Activity against Cotton aphid (Aphis gossypii)

The active compounds are formulated as a 300 ppm solution in 50:50 acetone:water and 100 ppm Kinetic™ surfactant.

Cotton plants at the cotyledon stage (one plant per pot) are infested by placing a heavily infested leaf from the main colony on top of each cotyledon. The aphids are allowed to transfer to the host plant overnight, and the leaf used to transfer the aphids is removed. The cotyledons are dipped in the test solution and allowed to dry. After 5 days, mortality counts are made.

7. Activity against Southern armyworm (Spodoptera eridania), 2nd instar larvae

The active compounds are formulated for testing the activity against insects and arachnids as a 300 ppm solution in a mixture of 35% acetone and water, which is diluted with water, if needed.

A Sieva lima bean leaf is dipped in the test solution and allowed to dry. The leaf is then placed in a petri dish containing a filter paper on the bottom and ten 2nd instar caterpillars. At 5 days, observations are made of mortality and reduced feeding.

8. Rice brown plant hopper (Nilaparvata lugens)

Rice seedlings were cleaned and washed 24 hours before spraying. The active compounds were formulated as a 300 ppm solution in 50:50 acetone:water and 0.1% vol/vol surfactant (EL 620) was added. Potted rice seedlings were sprayed with 5 ml test solution, air dried, placed in cages and inoculated with 10 adults. Treated rice plants were kept at 28-29° C. and relative humidity of 50-60%. Percent mortality was recorded after 72 hours.

In this test, compounds I-6 at 300 ppm showed over 80% mortality compared to 0% mortality of untreated controls.

9. Activity against Argentine ant (Linepithema humile), harvester ant (Pogonomyrmex californicus), acrobat ant (Crematogaster spp.), carpenter ant (Camponotus floridanus), fire ant (Solenopsis invicta), house fly (Musca domestica), stable fly (Stomoxys calcitrans), flesh fly (Sarcophaga sp.), yellowfever mosquito (Aedes aegyptii), house mosquito (Culex quinquefasciatus), malaria mosquito (Anopheles albimanus), German cockroach (Blattella Germanica), cat flea (Ctenocephalides felis), and brown dog tick (Rhipicephalus sanguineus) via glass contact

Glass vials were treated with 0.5 ml of a solution of 10 ppm of the active ingredient in acetone and allowed to dry. Insects or ticks were placed into each vial together with some food and moisture supply. The vials were kept at 22° C. and were observed for treatment effects at various time intervals.

In this test, compound I-2 at 10 ppm showed over 75% mortality against yellowfever mosquito compared to 0% mortality of untreated controls.

10. Activity against yellowfever mosquito (Aedes aegyptii), house mosquito (Culex quinquefasciatus) and malaria mosquito (Anopheles albimanus) larvae via water treatment

Well plates are used as test arenas. The active ingredient is dissolved in acetone and diluted with water to obtain the concentrations needed. The final solution, containing appr. 1% acetone, are placed into each well. Approximately 10 mosquito larvae (4^{th}-instars) in 1 ml water are added to each well. Larvae are fed one drop of liver powder each day. The dishes are covered and maintained at 22° C. Mortality is recorded daily and dead larvae and live or dead pupae are removed daily. At the end of the test remaining live larvae are recorded and percent mortality is calculated.

1-16. (canceled)

17. A dicyano alkanoic acid amide of formula I 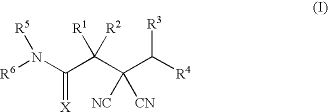
wherein
R¹ is hydrogen, C₁-C₁₀-alkyl, C₁-C₁₀-haloalkyl, C₂-C₁₀-alkenyl, C₂-C₁₀-haloalkenyl, C₂-C₁₀-alkynyl, or C₃-C₁₀-haloalkynyl;
R² is hydrogen, cyano, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₆-cycloalkyl, C₃-C₆-halocycloalkyl, C₁-C₁₀-alkoxy, or C₁-C₁₀-haloalkoxy;
R³ and R⁴ each independently are hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₁₀-cycloalkyl, C₃-C₁₀-halocycloalkyl, C₄-C₆-cycloalkenyl, C₄-C₆-halocycloalkenyl, or
R³ and R⁴ together can be C₂-C₁₀-alkylene, C₂-C₁₀-haloalkylene, C₄-C₁₀-alkenylene or C₄-C₁₀-haloalkenylene;
X is oxygen or sulfur;
R⁵ and R⁶ each independently are hydrogen, C₁-C₁₀-alkyl, which is unsubstituted or substituted with 1 to 3 substituents each independently selected from the group of cyano, nitro, hydroxy, NR⁷R⁸, C₁-C₁₀-alkoxy, C₁-C₁₀-haloalkoxy, C₁-C₁₀-alkylthio, C₁-C₁₀-haloalkylthio, C₃-C₆-cycloalkyl, C₃-C₆-halocycloalkyl and phenyl, wherein phenyl is unsubstituted or substituted with 1 to 5 substituents R^{a}; or
C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkylsulfinyl, C₁-C₁₀-haloalkylsulfinyl, C₁-C₁₀-alkylsulfonyl, C₁-C₁₀-haloalkylsulfonyl, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁷R⁸N—C(═O)—, phenylcarbonyl, benzylcarbonyl or phenyl wherein phenylcarbonyl, benzylcarbonyl and phenyl are unsubstituted or substituted with 1 to 5 substituents R^{a}; or
R⁵ and R⁶ each independently are a saturated, partially unsaturated or unsaturated 5- to 6-membered heterocyclic radical which may contain 1 to 4 heteroatoms selected from oxygen, nitrogen and sulfur and which radical may be bonded via a carbonyl (C(═O)) linkage, and which radical is unsubstituted or substituted with any combination of 1 to 4 groups R^{a};
R^{a} is halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₁-C₁₀-alkylcarbonyl, C₁-C₁₀-haloalkylcarbonyl or R⁷R⁸N—C(═O)—;
R⁷, R⁸ are each independently hydrogen, C₁-C₁₀-alkyl, C₁-C₁₀-haloalkyl, C₁-C₁₀-alkylcarbonyl, C₁-C₁₀-haloalkylcarbonyl, benzyl, phenyl, phenylcarbonyl, C₁-C₁₀-alkylsulfonyl, or C₁-C₁₀-haloalkylsulfonyl;
or the enantiomers or diastereomers or salts thereof.

18. The Dicyano alkanoic acid amide of formula I of claim 17 wherein X is oxygen.

19. The Dicyano alkanoic acid amide of formula I of claim 17 wherein R⁵ and R⁶ are each independently hydrogen, C₁-C₁₀-alkyl, which is unsubstituted or substituted by one substituent selected from the group consisting of cyano, nitro, C₁-C₁₀-alkoxy, C₁-C₁₀-alkylthio, phenyl and C₃-C₆-cycloalkyl; or C₁-C₁₀-haloalkyl, C₃-C₁₀-cycloalkyl, C₂-C₁₀-alkenyl, C₂-C₁₀-alkynyl, C₁-C₁₀-alkylcarbonyl, C₁-C₁₀-haloalkylcarbonyl, phenylcarbonyl, benzylcarbonyl or phenyl.

20. A process for the preparation of a dicyano alkanoic acid amide compound of formula I: 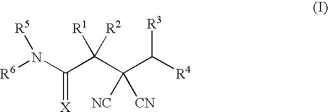
wherein
R¹ is hydrogen, C₁-C₁₀-alkyl, C₁-C₁₀-haloalkyl, C₂-C₁₀-alkenyl, C₂-C₁₀-haloalkenyl, C₂-C₁₀-alkynyl, or C₃-C₁₀-haloalkynyl;
R² is hydrogen, cyano, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₆-cycloalkyl, C₃-C₆-halocycloalkyl, C₁-C₁₀-alkoxy, or C₁-C₁₀-haloalkoxy;
R³ and R⁴ each independently are hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₁₀-cycloalkyl, C₃-C₁₀-halocycloalkyl, C₄-C₆-cycloalkenyl, C₄-C₆-halocycloalkenyl, or
R³ and R⁴ together can be C₂-C₁₀-alkylene, C₂-C₁₀-haloalkylene, C₄-C₁₀-alkenylene or C₄-C₁₀-haloalkenylene;
X is oxygen or sulfur;
R⁵ and R⁶ each independently are hydrogen, C₁-C₁₀-alkyl, which is unsubstituted or substituted with 1 to 3 substituents each independently selected from the group of cyano, nitro, hydroxy, NR⁷R⁸, C₁-C₁₀-alkoxy, C₁-C₁₀-haloalkoxy, C₁-C₁₀-alkylthio, C₁-C₁₀-haloalkylthio, C₃-C₆-cycloalkyl, C₃-C₆-halocycloalkyl and phenyl, wherein phenyl is unsubstituted or substituted with 1 to 5 substituents R^{a}; or
C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkylsulfinyl, C₁-C₁₀-haloalkylsulfinyl, C₁-C₁₀-alkylsulfonyl, C₁-C₁₀-haloalkylsulfonyl, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁷R⁸N—C(═O)—, phenylcarbonyl, benzylcarbonyl or phenyl wherein phenylcarbonyl, benzylcarbonyl and phenyl are unsubstituted or substituted with 1 to 5 substituents R^{a}; or
R⁵ and R⁶ each independently are a saturated, partially unsaturated or unsaturated 5- to 6-membered heterocyclic radical which may contain 1 to 4 heteroatoms selected from oxygen, nitrogen and sulfur and which radical may be bonded via a carbonyl (C(═O)) linkage, and which radical is unsubstituted or substituted with any combination of 1 to 4 groups R^{a};
R^{a} is halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₁-C₁₀-alkylcarbonyl, C₁-C₁₀-haloalkylcarbonyl or R⁷R⁸N—C(═O)—;
R⁷, R⁸ are each independently hydrogen, C₁-C₁₀-alkyl, C₁-C₁₀-haloalkyl, C₁-C₁₀-alkylcarbonyl, C₁-C₁₀-haloalkylcarbonyl, benzyl, phenyl, phenylcarbonyl, C₁-C₁₀-alkylsulfonyl, or C₁-C₁₀-haloalkylsulfonyl;
or the enantiomers or diastereomers or salts thereof; wherein the process comprises:
reacting compounds (II) with compound (III) in the presence of a base to give compounds (I), 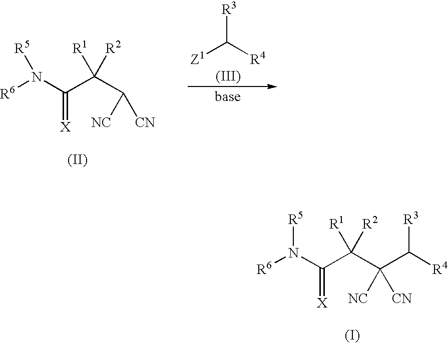
wherein X, R¹, R², R³, R⁴, R⁵ and R⁶ are as defined above for compounds of formula I and Z¹ represents a halogen atom, methanesulfonyl, trifluoromethanesulfonyl or toluenesulfonyl.

21. A method for combating insects, acarids or nematodes by contacting the insect, acarid or nematode or their food supply, habitat, breeding ground or their locus with a pesticidally effective amount of a dicyano alkanoic acid amide compound of formula I: 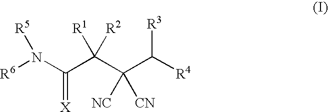
wherein
R¹ is hydrogen, C₁-C₁₀-alkyl, C₁-C₁₀-haloalkyl, C₂-C₁₀-alkenyl, C₂-C₁₀-haloalkenyl, C₂-C₁₀-alkynyl, or C₃-C₁₀-haloalkynyl;
R² is hydrogen, cyano, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₆-cycloalkyl, C₃-C₆-halocycloalkyl, C₁-C₁₀-alkoxy, or C₁-C₁₀-haloalkoxy;
R³ and R⁴ each independently are hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₁₀-cycloalkyl, C₃-C₁₀-halocycloalkyl, C₄-C₆-cycloalkenyl, C₄-C₆-halocycloalkenyl, or
R³ and R⁴ together can be C₂-C₁₀-alkylene, C₂-C₁₀-haloalkylene, C₄-C₁₀-alkenylene or C₄-C₁₀-haloalkenylene;
X is oxygen or sulfur;
R⁵ and R⁶ each independently are hydrogen, C₁-C₁₀-alkyl, which is unsubstituted or substituted with 1 to 3 substituents each independently selected from the group of cyano, nitro, hydroxy, NR⁷R⁸, C₁-C₁₀-alkoxy, C₁-C₁₀-haloalkoxy, C₁-C₁₀-alkylthio, C₁-C₁₀-haloalkylthio, C₃-C₆-cycloalkyl, C₃-C₆-halocycloalkyl and phenyl, wherein phenyl is unsubstituted or substituted with 1 to 5 substituents R^{a}; or
C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkylsulfinyl, C₁-C₁₀-haloalkylsulfinyl, C₁-C₁₀-alkylsulfonyl, C₁-C₁₀-haloalkylsulfonyl, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁷R⁸N—C(═O)—, phenylcarbonyl, benzylcarbonyl or phenyl wherein phenylcarbonyl, benzylcarbonyl and phenyl are unsubstituted or substituted with 1 to 5 substituents R^{a}; or
R⁵ and R⁶ each independently are a saturated, partially unsaturated or unsaturated 5- to 6-membered heterocyclic radical which may contain 1 to 4 heteroatoms selected from oxygen, nitrogen and sulfur and which radical may be bonded via a carbonyl (C(═O)) linkage, and which radical is unsubstituted or substituted with any combination of 1 to 4 groups R^{a};
R^{a} is halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₁-C₁₀-alkylcarbonyl, C₁-C₁₀-haloalkylcarbonyl or R⁷R⁸N—C(═O)—;
R⁷, R⁸ are each independently hydrogen, C₁-C₁₀-alkyl, C₁-C₁₀-haloalkyl, C₁-C₁₀-alkylcarbonyl, C₁-C₁₀-haloalkylcarbonyl, benzyl, phenyl, phenylcarbonyl, C₁-C₁₀-alkylsulfonyl, or C₁-C₁₀-haloalkylsulfonyl;
or the enantiomers or diastereomers or salts thereof.

22. A method for the control of insects, acarids or nematodes by contacting the insect, acarid or nematode or their food supply, habitat, breeding ground or their locus with a pesticidally effective amount of a dicyano alkanoic acid amide compound of formula I 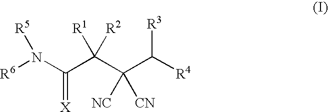
wherein
R¹ is hydrogen, C₁-C₁₀-alkyl, C₁-C₁₀-haloalkyl, C₂-C₁₀-alkenyl, C₂-C₁₀-haloalkenyl, C₂-C₁₀-alkynyl, or C₃-C₁₀-haloalkynyl;
R² is hydrogen, cyano, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₆-cycloalkyl, C₃-C₆-halocycloalkyl, C₁-C₁₀-alkoxy, or C₁-C₁₀-haloalkoxy;
R³ and R⁴ each independently are hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₁₀-cycloalkyl, C₃-C₁₀-halocycloalkyl, C₄-C₆-cycloalkenyl, C₄-C₆-halocycloalkenyl, or
R³ and R⁴ together can be C₂-C₁₀-alkylene, C₂-C₁₀-haloalkylene, C₄-C₁₀-alkenylene or C₄-C₁₀-haloalkenylene;
X is oxygen or sulfur;
R⁵ and R⁶ each independently are hydrogen, C₁-C₁₀-alkyl, which is unsubstituted or substituted with 1 to 3 substituents each independently selected from the group of cyano, nitro, hydroxy, NR⁷R⁸, C₁-C₁₀-alkoxy, C₁-C₁₀-haloalkoxy, C₁-C₁₀-alkylthio, C₁-C₁₀-haloalkylthio, C₃-C₆-cycloalkyl, C₃-C₆-halocycloalkyl and phenyl, wherein phenyl is unsubstituted or substituted with 1 to 5 substituents R^{a}; or
C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkylsulfinyl, C₁-C₁₀-haloalkylsulfinyl, C₁-C₁₀-alkylsulfonyl, C₁-C₁₀-haloalkylsulfonyl, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁷R⁸N—C(═O)—, phenylcarbonyl, benzylcarbonyl or phenyl wherein phenylcarbonyl, benzylcarbonyl and phenyl are unsubstituted or substituted with 1 to 5 substituents R^{a}; or
R⁵ and R⁶ each independently are a saturated, partially unsaturated or unsaturated 5- to 6-membered heterocyclic radical which may contain 1 to 4 heteroatoms selected from oxygen, nitrogen and sulfur and which radical may be bonded via a carbonyl (C(═O)) linkage, and which radical is unsubstituted or substituted with any combination of 1 to 4 groups R^{a};
R^{a} is halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₁-C₁₀-alkylthio, C₁-C₁₀ haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₁-C₁₀-alkylcarbonyl, C₁-C₁₀-haloalkylcarbonyl or R⁷R⁸N—C(═O)—;
R⁷, R⁸ are each independently hydrogen, C₁-C₁₀-alkyl, C₁-C₁₀-haloalkyl, C₁-C₁₀-alkylcarbonyl, C₁-C₁₀-haloalkylcarbonyl, benzyl, phenyl, phenylcarbonyl, C₁-C₁₀-alkylsulfonyl, or C₁-C₁₀-haloalkylsulfonyl;
or the enantiomers or diastereomers or salts thereof, or compositions comprising at least one compound thereof.

23. A method of protecting growing plants from attack or infestation by insects, acarids or nematodes by applying to the foliage of the plants, or to the soil or water in which they are growing, a pesticidally effective amount of a dicyano alkanoic acid amide compound of formula I: 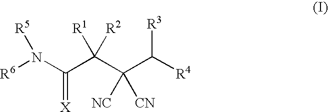
wherein
R¹ is hydrogen, C₁-C₁₀-alkyl, C₁-C₁₀-haloalkyl, C₂-C₁₀-alkenyl, C₂-C₁₀-haloalkenyl, C₂-C₁₀-alkynyl, or C₃-C₁₀-haloalkynyl;
R² is hydrogen, cyano, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₆-cycloalkyl, C₃-C₆-halocycloalkyl, C₁-C₁₀-alkoxy, or C₁-C₁₀-haloalkoxy;
R³ and R⁴ each independently are hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₁₀-cycloalkyl, C₃-C₁₀-halocycloalkyl, C₄-C₆-cycloalkenyl, C₄-C₆-halocycloalkenyl, or
R³ and R⁴ together can be C₂-C₁₀-alkylene, C₂-C₁₀-haloalkylene, C₄-C₁₀-alkenylene or C₄-C₁₀-haloalkenylene;
X is oxygen or sulfur;
R⁵ and R⁶ each independently are hydrogen, C₁-C₁₀-alkyl, which is unsubstituted or substituted with 1 to 3 substituents each independently selected from the group of cyano, nitro, hydroxy, NR⁷R⁸, C₁-C₁₀-alkoxy, C₁-C₁₀-haloalkoxy, C₁-C₁₀-alkylthio, C₁-C₁₀-haloalkylthio, C₃-C₆-cycloalkyl, C₃-C₆-halocycloalkyl and phenyl, wherein phenyl is unsubstituted or substituted with 1 to 5 substituents R^{a}; or
C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkylsulfinyl, C₁-C₁₀-haloalkylsulfinyl, C₁-C₁₀-alkylsulfonyl, C₁-C₁₀-haloalkylsulfonyl, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁷R⁸N—C(═O)—, phenylcarbonyl, benzylcarbonyl or phenyl wherein phenylcarbonyl, benzylcarbonyl and phenyl are unsubstituted or substituted with 1 to 5 substituents R^{a}; or
R⁵ and R⁶ each independently are a saturated, partially unsaturated or unsaturated 5- to 6-membered heterocyclic radical which may contain 1 to 4 heteroatoms selected from oxygen, nitrogen and sulfur and which radical may be bonded via a carbonyl (C(═O)) linkage, and which radical is unsubstituted or substituted with any combination of 1 to 4 groups R^{a};
R^{a} is halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₁-C₁₀-alkylcarbonyl, C₁-C₁₀-haloalkylcarbonyl or R⁷R⁸N—C(═O)—;
R⁷, R⁸ are each independently hydrogen, C₁-C₁₀-alkyl, C₁-C₁₀-haloalkyl, C₁-C₁₀-alkylcarbonyl, C₁-C₁₀-haloalkylcarbonyl, benzyl, phenyl, phenylcarbonyl, C₁-C₁₀-alkylsulfonyl, or C₁-C₁₀-haloalkylsulfonyl;
or the enantiomers or diastereomers or salts thereof, or compositions comprising at least one compound thereof.

24. A method for treating, controlling, preventing or protecting animals against infestation or infection by parasites which comprises orally, topically or parenterally administering or applying to the animals a parasiticidally effective amount of a dicyano alkanoic acid amide compound of formula I: 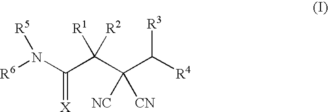
wherein
R¹ is hydrogen, C₁-C₁₀-alkyl, C₁-C₁₀-haloalkyl, C₂-C₁₀-alkenyl, C₂-C₁₀-haloalkenyl, C₂-C₁₀-alkynyl, or C₃-C₁₀-haloalkynyl;
R² is hydrogen, cyano, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₆-cycloalkyl, C₃-C₆-halocycloalkyl, C₁-C₁₀-alkoxy, or C₁-C₁₀-haloalkoxy;
R³ and R⁴ each independently are hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₁₀-cycloalkyl, C₃-C₁₀-halocycloalkyl, C₄-C₆-cycloalkenyl, C₄-C₆-halocycloalkenyl, or
R³ and R⁴ together can be C₂-C₁₀-alkylene, C₂-C₁₀-haloalkylene, C₄-C₁₀alkenylene or C₄-C₁₀-haloalkenylene;
X is oxygen or sulfur;
R⁵ and R⁶ each independently are hydrogen, C₁-C₁₀-alkyl, which is unsubstituted or substituted with 1 to 3 substituents each independently selected from the group of cyano, nitro, hydroxy, NR⁷R⁸, C₁-C₁₀-alkoxy, C₁-C₁₀-haloalkoxy, C₁-C₁₀-alkylthio, C₁-C₁₀-haloalkylthio, C₃-C₆-cycloalkyl, C₃-C₆-halocycloalkyl and phenyl, wherein phenyl is unsubstituted or substituted with 1 to 5 substituents R^{a}; or
C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkylsulfinyl, C₁-C₁₀-haloalkylsulfinyl, C₁-C₁₀-alkylsulfonyl, C₁-C₁₀-haloalkylsulfonyl, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁷R⁸N—C(═O)—, phenylcarbonyl, benzylcarbonyl or phenyl wherein phenylcarbonyl, benzylcarbonyl and phenyl are unsubstituted or substituted with 1 to 5 substituents R^{a}; or
R⁵ and R⁶ each independently are a saturated, partially unsaturated or unsaturated 5- to 6-membered heterocyclic radical which may contain 1 to 4 heteroatoms selected from oxygen, nitrogen and sulfur and which radical may be bonded via a carbonyl (C(═O)) linkage, and which radical is unsubstituted or substituted with any combination of 1 to 4 groups R^{a};
R^{a} is halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₁-C₁₀-alkylcarbonyl, C₁-C₁₀-haloalkylcarbonyl or R⁷R⁸N—C(═O)—;
R⁷, R⁸ are each independently hydrogen, C₁-C₁₀-alkyl, C₁-C₁₀-haloalkyl, C₁-C₁₀-alkylcarbonyl, C₁-C₁₀-haloalkylcarbonyl, benzyl, phenyl, phenylcarbonyl, C₁-C₁₀-alkylsulfonyl, or C₁-C₁₀-haloalkylsulfonyl;
or the enantiomers or diastereomers or salts thereof, their enantiomers or veterinarily acceptable salts, or compositions comprising at least one compound thereof.

25. A method for the preparation of a composition for treating, controlling, preventing or protecting animals against infestation or infection by parasites wherein the method comprises a parasiticidally effective amount of a dicyano alkanoic acid amide compound of formula I: 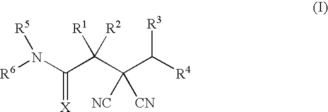
wherein
R¹ is hydrogen, C₁-C₁₀-alkyl, C₁-C₁₀-haloalkyl, C₂-C₁₀-alkenyl, C₂-C₁₀-haloalkenyl, C₂-C₁₀-alkynyl, or C₃-C₁₀-haloalkynyl;
R² is hydrogen, cyano, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₆-cycloalkyl, C₃-C₆-halocycloalkyl, C₁-C₁₀-alkoxy, or C₁-C₁₀-haloalkoxy;
R³ and R⁴ each independently are hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₁₀-cycloalkyl, C₃-C₁₀-halocycloalkyl, C₄-C₆-cycloalkenyl, C₄-C₆-halocycloalkenyl, or
R³ and R⁴ together can be C₂-C₁₀-alkylene, C₂-C₁₀-haloalkylene, C₄-C₁₀-alkenylene or C₄-C₁₀-haloalkenylene;
X is oxygen or sulfur;
R⁵ and R⁶ each independently are hydrogen, C₁-C₁₀-alkyl, which is unsubstituted or substituted with 1 to 3 substituents each independently selected from the group of cyano, nitro, hydroxy, NR⁷R⁸, C₁-C₁₀-alkoxy, C₁-C₁₀-haloalkoxy, C₁-C₁₀-alkylthio, C₁-C₁₀-haloalkylthio, C₃-C₆-cycloalkyl, C₃-C₆-halocycloalkyl and phenyl, wherein phenyl is unsubstituted or substituted with 1 to 5 substituents R^{a}; or
C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkylsulfinyl, C₁-C₁₀-haloalkylsulfinyl, C₁-C₁₀-alkylsulfonyl, C₁-C₁₀-haloalkylsulfonyl, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁷R⁸N—C(═O)—, phenylcarbonyl, benzylcarbonyl or phenyl wherein phenylcarbonyl, benzylcarbonyl and phenyl are unsubstituted or substituted with 1 to 5 substituents R^{a}; or
R⁵ and R⁶ each independently are a saturated, partially unsaturated or unsaturated 5- to 6-membered heterocyclic radical which may contain 1 to 4 heteroatoms selected from oxygen, nitrogen and sulfur and which radical may be bonded via a carbonyl (C(═O)) linkage, and which radical is unsubstituted or substituted with any combination of 1 to 4 groups R^{a};
R^{a} is halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₁-C₁₀-alkylcarbonyl, C₁-C₁₀-haloalkylcarbonyl or R⁷R⁸N—C(═O)—;
R⁷, R⁸ are each independently hydrogen, C₁-C₁₀-alkyl, C₁-C₁₀-haloalkyl, C₁-C₁₀-alkylcarbonyl, C₁-C₁₀-haloalkylcarbonyl, benzyl, phenyl, phenylcarbonyl, C₁-C₁₀-alkylsulfonyl, or C₁-C₁₀-haloalkylsulfonyl;
or the enantiomers or diastereomers or salts thereof, or their enantiomers or veterinary acceptable salts.

26. A veterinary composition comprising a parasiticidally active amount of a compound of formula I: 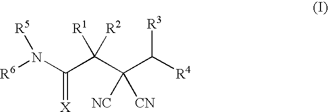
wherein
R¹ is hydrogen, C₁-C₁₀-alkyl, C₁-C₁₀-haloalkyl, C₂-C₁₀-alkenyl, C₂-C₁₀-haloalkenyl, C₂-C₁₀-alkynyl, or C₃-C₁₀-haloalkynyl;
R² is hydrogen, cyano, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₆-cycloalkyl, C₃-C₆-halocycloalkyl, C₁-C₁₀-alkoxy, or C₁-C₁₀-haloalkoxy;
R³ and R⁴ each independently are hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₁₀-cycloalkyl, C₃-C₁₀-halocycloalkyl, C₄-C₆-cycloalkenyl, C₄-C₆-halocycloalkenyl, or
R³ and R⁴ together can be C₂-C₁₀-alkylene, C₂-C₁₀-haloalkylene, C₄-C₁₀-alkenylene or C₄-C₁₀-haloalkenylene;
X is oxygen or sulfur;
R⁵ and R⁶ each independently are hydrogen, C₁-C₁₀-alkyl, which is unsubstituted or substituted with 1 to 3 substituents each independently selected from the group of cyano, nitro, hydroxy, NR⁷R⁸, C₁-C₁₀-alkoxy, C₁-C₁₀-haloalkoxy, C₁-C₁₀-alkylthio, C₁-C₁₀-haloalkylthio, C₃-C₆-cycloalkyl, C₃-C₆-halocycloalkyl and phenyl, wherein phenyl is unsubstituted or substituted with 1 to 5 substituents R^{a}; or
C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkylsulfinyl, C₁-C₁₀-haloalkylsulfinyl, C₁-C₁₀-alkylsulfonyl, C₁-C₁₀-haloalkylsulfonyl, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁷R⁸N—C(═O)—, phenylcarbonyl, benzylcarbonyl or phenyl wherein phenylcarbonyl, benzylcarbonyl and phenyl are unsubstituted or substituted with 1 to 5 substituents R^{a}; or
R⁵ and R⁶ each independently are a saturated, partially unsaturated or unsaturated 5- to 6-membered heterocyclic radical which may contain 1 to 4 heteroatoms selected from oxygen, nitrogen and sulfur and which radical may be bonded via a carbonyl (C(═O)) linkage, and which radical is unsubstituted or substituted with any combination of 1 to 4 groups R^{a};
R^{a} is halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₁-C₁₀-alkylcarbonyl, C₁-C₁₀-haloalkylcarbonyl or R⁷R⁸N—C(═O)—;
R⁷, R⁸ are each independently hydrogen, C₁-C₁₀-alkyl, C₁-C₁₀-haloalkyl, C₁-C₁₀-alkylcarbonyl, C₁-C₁₀-haloalkylcarbonyl, benzyl, phenyl, phenylcarbonyl, C₁-C₁₀-alkylsulfonyl, or C₁-C₁₀-haloalkylsulfonyl;
or the enantiomers or diastereomers or salts thereof and a veterinary acceptable carrier.

27. An agricultural composition comprising a pesticidally active amount of a compound of formula I: 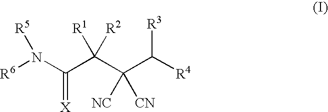
wherein
R¹ is hydrogen, C₁-C₁₀-alkyl, C₁-C₁₀-haloalkyl, C₂-C₁₀-alkenyl, C₂-C₁₀-haloalkenyl, C₂-C₁₀-alkynyl, or C₃-C₁₀-haloalkynyl;
R² is hydrogen, cyano, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₆-cycloalkyl, C₃-C₆-halocycloalkyl, C₁-C₁₀-alkoxy, or C₁-C₁₀-haloalkoxy;
R³ and R⁴ each independently are hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₁₀-cycloalkyl, C₃-C₁₀-halocycloalkyl, C₄-C₆-cycloalkenyl, C₄-C₆-halocycloalkenyl, or
R³ and R⁴ together can be C₂-C₁₀-alkylene, C₂-C₁₀-haloalkylene, C₄-C₁₀-alkenylene or C₄-C₁₀-haloalkenylene;
X is oxygen or sulfur;
R⁵ and R⁶ each independently are hydrogen, C₁-C₁₀-alkyl, which is unsubstituted or substituted with 1 to 3 substituents each independently selected from the group of cyano, nitro, hydroxy, NR⁷R⁸, C₁-C₁₀-alkoxy, C₁-C₁₀-haloalkoxy, C₁-C₁₀-alkylthio, C₁-C₁₀-haloalkylthio, C₃-C₆-cycloalkyl, C₃-C₆-halocycloalkyl and phenyl, wherein phenyl is unsubstituted or substituted with 1 to 5 substituents R^{a}; or
C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkylsulfinyl, C₁-C₁₀-haloalkylsulfinyl, C₁-C₁₀-alkylsulfonyl, C₁-C₁₀-haloalkylsulfonyl, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁷R⁸N—C(═O)—, phenylcarbonyl, benzylcarbonyl or phenyl wherein phenylcarbonyl, benzylcarbonyl and phenyl are unsubstituted or substituted with 1 to 5 substituents R^{a}; or
R⁵ and R⁶ each independently are a saturated, partially unsaturated or unsaturated 5- to 6-membered heterocyclic radical which may contain 1 to 4 heteroatoms selected from oxygen, nitrogen and sulfur and which radical may be bonded via a carbonyl (C(═O)) linkage, and which radical is unsubstituted or substituted with any combination of 1 to 4 groups R^{a};
R^{a} is halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₁-C₁₀-alkylcarbonyl, C₁-C₁₀-haloalkylcarbonyl or R⁷R⁸N—C(═O)—;
R⁷, R⁸ are each independently hydrogen, C₁-C₁₀-alkyl, C₁-C₁₀-haloalkyl, C₁-C₁₀-alkylcarbonyl, C₁-C₁₀-haloalkylcarbonyl, benzyl, phenyl, phenylcarbonyl, C₁-C₁₀-alkylsulfonyl, or C₁-C₁₀-haloalkylsulfonyl;
or the enantiomers or diastereomers or salts thereof and an agriculturally acceptable carrier.

28. A method for the protection of seeds from soil insects and of the seedlings'roots and shoots from soil and foliar insects comprising contacting the seeds before sowing and/or after pregermination with at least one dicyano alkanoic acid amide compound of the formula I: 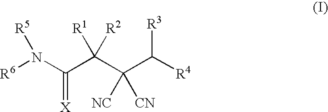
wherein
R¹ is hydrogen, C₁-C₁₀-alkyl, C₁-C₁₀-haloalkyl, C₂-C₁₀-alkenyl, C₂-C₁₀-haloalkenyl, C₂-C₁₀-alkynyl, or C₃-C₁₀-haloalkynyl;
R² is hydrogen, cyano, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₆-cycloalkyl, C₃-C₆-halocycloalkyl, C₁-C₁₀-alkoxy, or C₁-C₁₀-haloalkoxy;
R³ and R⁴ each independently are hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₁₀-cycloalkyl, C₃-C₁₀-halocycloalkyl, C₄-C₆-cycloalkenyl, C₄-C₆-halocycloalkenyl, or
R³ and R⁴ together can be C₂-C₁₀-alkylene, C₂-C₁₀-haloalkylene, C₄-C₁₀-alkenylene or C₄-C₁₀-haloalkenylene;
X is oxygen or sulfur;
R⁵ and R⁶ each independently are hydrogen, C₁-C₁₀-alkyl, which is unsubstituted or substituted with 1 to 3 substituents each independently selected from the group of cyano, nitro, hydroxy, NR⁷R⁸, C₁-C₁₀-alkoxy, C₁-C₁₀-haloalkoxy, C₁-C₁₀-alkylthio, C₁-C₁₀-haloalkylthio, C₃-C₆-cycloalkyl, C₃-C₆-halocycloalkyl and phenyl, wherein phenyl is unsubstituted or substituted with 1 to 5 substituents R^{a}; or
C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkylsulfinyl, C₁-C₁₀-haloalkylsulfinyl, C₁-C₁₀-alkylsulfonyl, C₁-C₁₀-haloalkylsulfonyl, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁷R⁸N—C(═O)—, phenylcarbonyl, benzylcarbonyl or phenyl wherein phenylcarbonyl, benzylcarbonyl and phenyl are unsubstituted or substituted with 1 to 5 substituents R^{a}; or
R⁵ and R⁶ each independently are a saturated, partially unsaturated or unsaturated 5- to 6-membered heterocyclic radical which may contain 1 to 4 heteroatoms selected from oxygen, nitrogen and sulfur and which radical may be bonded via a carbonyl (C(═O)) linkage, and which radical is unsubstituted or substituted with any combination of 1 to 4 groups R^{a};
R^{a} is halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₁-C₁₀-alkylcarbonyl, C₁-C₁₀-haloalkylcarbonyl or R⁷R⁸N—C(═O)—;
R⁷, R⁸ are each independently hydrogen, C₁-C₁₀-alkyl, C₁-C₁₀-haloalkyl, C₁-C₁₀-alkylcarbonyl, C₁-C₁₀-haloalkylcarbonyl, benzyl, phenyl, phenylcarbonyl, C₁-C₁₀-alkylsulfonyl, or C₁-C₁₀-haloalkylsulfonyl;
or the enantiomers or diastereomers or salts thereof, or a composition comprising at least one compound thereof.

29. The method according to claim 28, wherein the dicyano alkanoic acid amide compound of the formula I is applied in an amount of from 100 mg to 10 kg per 100 kg of seeds.

30. The method according to claim 28, wherein of the resulting plant's roots and shoots are protected.

31. The method according to claim 28, wherein the resulting plant's shoots are protected from aphids.

32. A seed comprising a dicyano alkanoic acid amide compound of the formula I: 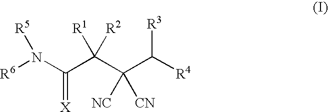
wherein
R¹ is hydrogen, C₁-C₁₀-alkyl, C₁-C₁₀-haloalkyl, C₂-C₁₀-alkenyl, C₂-C₁₀-haloalkenyl, C₂-C₁₀-alkynyl, or C₃-C₁₀-haloalkynyl;
R² is hydrogen, cyano, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₆-cycloalkyl, C₃-C₆-halocycloalkyl, C₁-C₁₀-alkoxy, or C₁-C₁₀-haloalkoxy;
R³ and R⁴ each independently are hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₁₀-cycloalkyl, C₃-C₁₀-halocycloalkyl, C₄-C₆-cycloalkenyl, C₄-C₆-halocycloalkenyl, or
R³ and R⁴ together can be C₂-C₁₀-alkylene, C₂-C₁₀-haloalkylene, C₄-C₁₀-alkenylene or C₄-C₁₀-haloalkenylene;
X is oxygen or sulfur;
R⁵ and R⁶ each independently are hydrogen, C₁-C₁₀-alkyl, which is unsubstituted or substituted with 1 to 3 substituents each independently selected from the group of cyano, nitro, hydroxy, NR⁷R⁸, C₁-C₁₀-alkoxy, C₁-C₁₀-haloalkoxy, C₁-C₁₀-alkylthio, C₁-C₁₀-haloalkylthio, C₃-C₆-cycloalkyl, C₃-C₆-halocycloalkyl and phenyl, wherein phenyl is unsubstituted or substituted with 1 to 5 substituents R^{a}; or
C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkylsulfinyl, C₁-C₁₀-haloalkylsulfinyl, C₁-C₁₀-alkylsulfonyl, C₁-C₁₀-haloalkylsulfonyl, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁷R⁸N—C(═O)—, phenylcarbonyl, benzylcarbonyl or phenyl wherein phenylcarbonyl, benzylcarbonyl and phenyl are unsubstituted or substituted with 1 to 5 substituents R^{a}; or
R⁵ and R⁶ each independently are a saturated, partially unsaturated or unsaturated 5- to 6-membered heterocyclic radical which may contain 1 to 4 heteroatoms selected from oxygen, nitrogen and sulfur and which radical may be bonded via a carbonyl (C(═O)) linkage, and which radical is unsubstituted or substituted with any combination of 1 to 4 groups R^{a};
R^{a} is halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₁-C₁₀-alkylcarbonyl, C₁-C₁₀-haloalkylcarbonyl or R⁷R⁸N—C(═O)—;
R⁷, R⁸ are each independently hydrogen, C₁-C₁₀-alkyl, C₁-C₁₀-haloalkyl, C₁-C₁₀-alkylcarbonyl, C₁-C₁₀-haloalkylcarbonyl, benzyl, phenyl, phenylcarbonyl, C₁-C₁₀-alkylsulfonyl, or C₁-C₁₀-haloalkylsulfonyl;
or the enantiomers or diastereomers or agriculturally useful salts thereof, in an amount of from 0.1 g to 10 kg per 100 kg of seed.
